(19) United States
(12) Patent Application Publication
(10) Pub. No.: US 2012/0178105 A1
(43) Pub. Date: Jul. 12, 2012

(54) DETECTION OF GLOBOTRIAOSYLCERAMIDE (GLC) IN HUMAN URINE SAMPLES USING AN ANTIBODY SANDWICH

(75) Inventors: PAUL F. HALLORAN, Auburn, MA (US); Michael A. Holland, Danvers, MA (US); Thomas Liponis, Milbury, MA (US); Thomas L. Pisani, Winchester, MA (US)

(73) Assignee: Genzyme Corporation, Cambridge, MA (US)

(21) Appl. No.: 12/987,826

(22) Filed: Jan. 10, 2011

(51) Int. Cl. G01N 33/566

(52) U.S. Cl. 435/7.92; 436/501

(57) ABSTRACT

Applicant has developed an assay for the detection of GL3 in human samples using a sandwich based immunoassay in which utilizes a pair of GL3 specific monoclonal antibodies, one for capture and one for detection, to create an antibody “sandwich” around the GL3 ligand. To further increase sensitivity, Applicant has modified traditional sandwich based assays by complexing the capture antibody with GL3 before adding the sample or detector antibody, providing an inhibition based assay.

### BACKGROUND

Fabry's disease is a rare X-linked recessive lysosomal storage disease. A deficiency of the enzyme alpha galactosidase A, due to mutation, causes a glycolipid known as globotriaosylceramide (also known as GL3, GB3, CTH, trihexosyl ceramide, ceramide trihexosamide) to accumulate in several cell types, including in the endothelial, perithelial, and smooth muscle cells of blood vessels. This progressive accumulation leads to an impairment of proper cellular function. Desnick et al. (1995) in The Metabolic Basis of Inherited Disease (Scriver et al. Eds) pg 2741-2784, McGraw Hill, NY).

Virtually all males with a mutation in the gene encoding enzyme alpha galactosidase A develop Fabry's disease and are likely to express some or many of the classic Fabry symptoms. However, symptoms in women with a mutation in the gene encoding enzyme alpha galactosidase A range from none (in asymptomatic carriers) to very serious manifestations similar to those seen in males. Often, the severity correlates to the amount of alpha galactosidase A enzyme produced in the body. Females with the faulty gene can have anywhere from near-normal levels of alpha galactosidase A to no active enzyme. Males, on the other hand, usually have little or no active alpha galactosidase A and are more likely to experience more severe symptoms than females.

Diagnosis of Fabry's disease can be challenging since signs and symptoms associated with Fabry's disease are widely varied, and may mimic those of other disorders. Despite being an X-linked disorder, some females may express varying degrees of clinical manifestations. Moreover, there are variants of Fabry's disease that do not present with classical signs and symptoms. Atypical variants have residual plasma alpha galactosidase A levels (1% to 30% of normal) and present much later in life than patients with classical Fabry's disease. WO2008075959A1.

The predominant storage product in Fabry disease is the major natural substrate for α-galactosidase A, Gb3 [Gal(α1→4)Gal(β1→4)Glc(β1→1′)Cer], also called CTH or GL3. GL3 is a neutral glycolipid and consists of a family of isoforms arising from heterogeneity in the fatty acid component of the ceramide, Mills et al. J. Inherit. Metabolic Dis. (2005) 28:35-48.

The level of storage products in the urine and plasma is elevated in most, but not all, patients with Fabry's disease. The elevation reflects the clinical severity and progression of the disease and may be used to monitor the progress of the disease and effect of treatment. Bryan Winchester Chapter 12 pages 455-457, Prenatal diagnosis of disorders of lipid metabolism Genetic Disorders and the Fetus: Diagnosis, Prevention and Treatment, 6th Edition, Aubrey Milunsky (Editor), Jeff Milunsky (Editor) ISBN: 978-1-4051-9087-9, January 2010, Wiley-Blackwell. Earlier detection and diagnosis of Fabry disease would provide the opportunity for effective treatment, such as enzyme replacement therapy for alpha galactosidase A, before the disease has progressed to the point where organ dysfunction or failure has occurred.

Desnick et al. ((1970) Journal of Lipids Research 11:31-37) demonstrated the relative abundance of GL3 in the urine of Fabry patients relative to the very low levels detected in the urine of normal control individuals. To ascertain the relative levels of GL3 in urine, Desnick used lengthy techniques including lipid extraction, glycolipid isolation, oligosaccharide hydrolysis, and quantitation of the liberated monosaccharides by gas liquid chromatography (GLC) or high-pressure liquid chromatography (HPLC).

Bryan Winchester and Elisabeth Young¹ provide methods of quantitative determination of non-derivatized GL3 in plasma and urine by liquid chromatography in conjunction with electrospray ionization tandem mass spectrometry with the aid of internal standards C-17-GL3, [d4]C16- and [d47]C24-isoforms of GL3 and [d35]C18-GL3. Winchester et al.² report the concentration of total GL3 is obtained by adding the concentrations of the individual isoforms of GL3 in order to establish reference ranges for total GL3 in plasma and urine from Fabry hemizygotes, heterozygotes and normal controls. Winchester et al. further detail that plasma levels of GL3 from classic hemizygotes range from 4.3 to 27.6 ug/ml GL3, while plasma levels of GL3 from heterozygotes range from 4.4-12.0 ug/ml GL3, compared to plasma levels of GL3 from controls which range from 3.6-7.5 ug/ml GL3. Winchester et al. also detail that urinary levels of GL3 from classic hemizygotes range from 0.12-2.80 mg GL3/mmol Creatinine (CR), while urinary levels from heterozygotes range from 0.02-0.37 mg GL3/mmol CR, compared to urinary levels from controls which range from 0.01-0.03 mg GL3/mmol CR. ¹ Mills and Young, Chapter 18: Biochemical and genetic diagnosis of Fabry disease, Fabry disease: Perspectives from 5 years of FOS, Mehta, Atul; Beck, Michael; Sunder-Plassmann, Gere, editors, Oxford (UK): Oxford PharmaGenesis Ltd.; c2006, citing Mills et al. FEBS Lett. 2002; 515:171-6, and Fauler et al. Rapid Commun. Mass Spectrum. (2005): 19:1499-506. ² Mills and Young, ibid, citing Mills et al. J. Inherit. Metabolic Dis. (2005) 28:35-48; Mills et al. Eur. J. Pediatr. 2004; 163:595-603; and Young et al. Acta Pediatr Suppl. (2005) 447-51-4.

U.S. Pat. No. 7,563,591 (Chamoles) discloses an assay for determining the activity of lysosomal enzymes present in dried bodily fluids, such as blood, where, for example, the activity of a lysosomal enzyme is measured in a blood spot formed by placing blood on a porous surface material and drying. The patent notes that since the surface material does not interfere with the enzymatic activity determination, it does not need to be removed from sample solution during testing. The patent describes combining an eluent for releasing the assayed lysosomal enzyme from the dried sample, an incubation buffer and at least one substrate capable of reacting with the lysosomal enzyme with the dried blood spot, generating at least one enzyme product. The enzyme product is then measured to determine the activity of the lysosomal enzyme. Stated advantages of this method include the small amount of test sample required and the extended period of time for which a dried sample can be stored without losing its diagnostic value.

Though the Chamoles test is described as being accurate, there is a need for an assay for GL3 in bodily fluids and/or tissues which is faster, requires fewer procedural steps, and which is more sensitive for detecting or diagnosing Fabry's Disease, as well as for monitoring the effectiveness of treatment or disease progression.

Antibody technology is a well established technology for detecting ligands in bodily tissues and fluids. However, the efficacy of antibody technology is limited to the specificity, affinity and avidity of the antibodies to the antigen of interest. Several antibodies which have been produced against various glycolipids, and GL3 in particular, individually tend to have low binding affinities and/or are not specific for both the oligosaccharide and lipid moieties of GL3. See Zeidner et al. 1999 Analytical Biochemistry 267:104-113.

Rapid test devices for detecting ligands in body fluids are known in the literature. Typically, a rapid test device is designed to detect levels of ligands in body fluids, and/or other samples, using a minimal number of procedural steps which can be performed by an untrained person. Ideally, rapid test devices should yield reliable results with an acceptable degree of sensitivity or specificity. Most rapid test devices contain an interior permeable material, e.g., glass fiber, capable of transporting an aqueous solution by capillary action, wicking, or simple wetting. A lateral flow assay is an example of a rapid test device, and several embodiments of lateral flow assays known in the art are described below:

The lateral flow device described in U.S. Pat. No. 5,714,389 contains a “test site” where a first protein, e.g., antibody, having a binding site specific to a first epitope of the ligand of interest is located, immobilized, e.g., bound to the permeable material or to latex particles entrapped in or bonded to the permeable material. The test site is in fluid communication with the liquid flow path, e.g., of the sample which moves from its site of application to the test site. Also intricate to the use of the device is a conjugate comprising a second protein, e.g., an antibody to the ligand, where the second protein is bound to colored particles such as a metal sol or colloid, preferably gold.

In the sandwich technique, the conjugate is mixed with the ligand of a sample to form a complex in a liquid, which is then transported by diffusion along a flow path to the test site. At the test site, the ligand bound with the conjugate reacts with the immobilized first binding protein to form a “sandwich” of the first protein, ligand, second protein, and colored particles. This sandwich complex is progressively produced at the test site as sample continuously passes by, filling the reservoir. As more and more conjugate is immobilized, the colored particles aggregate at the test site and become visible through the window, indicating the presence of ligand in the liquid sample.

In the case of the competitive technique, the second protein of the conjugate bound to colored particles can be for, example, an analog of the ligand, or an authentic sample of the ligand itself, a fraction thereof which has comparable affinity for the first protein. Thus, this conjugate binds to the first protein in competition with the ligand. As the liquid sample containing the conjugate is transported by diffusion along a flow path to the test site, the ligand of the sample, if any, and the conjugate compete for sites of attachment to the first protein. If no ligand is present, colored particles aggregate at the test site, and the presence of color indicates the absence of detectable levels of ligand in the sample. If ligand is present, the amount of conjugate which binds at the test site is reduced, and no color, or a paler color, develops.

Color development at the test site may be compared with the color of one or more standards or internal controls to determine whether the development of color is a true indication of the presence or absence of the ligand, or an artifact caused by nonspecific absorption.

The sensitivity of lateral flow device assays is frequently reduced, however, by the presence or formation in the sample of undesirable solid components which block the passage of labeled ligand to the detection zone. With the goal of increasing the sensitivity of the results obtained through lateral flow assays, U.S. Pat. No. 5,559,041 teaches the application of filters through which the sample is passed as it is being transported by diffusion along a flow path, but before it contacts the test site. By incorporating at least one filter element before the assay indicia zone, an increase in sensitivity is achieved as compared to previous migration type assays. The filter, which preferably has been treated to reduce any inherent hydrophobicity, traps unwanted components in the fluid sample and allows unimpeded passage of labeled ligand. Thus, a proportionately greater amount of ligand binds to the assay indicia zone, and more accurate assay results are achieved.

U.S. Pat. No. 5,559,041 additionally discloses that by selecting a membrane with the appropriate texture and pore size, a second filter element can act as a controlled cell lysing system. For example in an assay performed on a sample of whole blood it is advantageous to select as the second filter element a membrane which would maintain the integrity of whole blood cells while serum migrates through. This prevents the discoloration associated with blood cell lysis from spreading into the assay indicia zone.

In one category of lateral flow assays, the conjugate comprising a second protein, e.g., an antibody to the ligand, where the second protein is bound to a label, e.g., colored particles such as a metal sol or colloid, is premixed with the sample as described above in U.S. Pat. No. 5,714,389. In another category of a lateral flow assay, the conjugate is reversibly attached to the permeable material/porous carrier in preserved form, e.g., lyophilized, at a site along the flow path between the sample inlet and the test site, as described in U.S. Pat. No. 5,602,040. That is, in this second category of lateral flow assays, a labeled conjugate/specific binding reagent becomes freely mobile within the porous carrier when in the moist state, and can migrate with the sample flow. The mobility can be facilitated by a material comprising sugar, in an amount effective to reduce interaction between the carrier and the labeled reagent.

A lateral flow assay can be modified to contain an assay chamber as described in U.S. Pat. No. 7,666,614, providing the advantage of not requiring that the sample be transferred to the apparatus until after extraction of the ligand. In the assay chamber, which is separate from the lateral flow immunochromatographic device; the ligand is extracted from said sample with a liquid extraction solution. The liquid extract of the assay chamber is then connected to the sample receiving region of said lateral flow immunochromatographic device, allowing the liquid extract to flow through the reaction site(s) and then through said capture site(s), without further addition of reagents or manipulation of said sample, enabling detection of the presence or absence in the sample of the ligand of interest.

The lateral flow assay disclosed in U.S. Pat. No. 7,144,742 provides for visually quantitating ligands of both high and low molecular weight. The lateral flow assay of U.S. Pat. No. 7,144,742 contains a lateral flow matrix which defines a flow path and which comprises in series, a sample receiving zone, a labeling zone, and one or more serially oriented capture zones. The labeling zone of the porous material comprises a reversibly bound conjugate comprising a second protein, which is complementary to the ligand, e.g., antibody which binds the ligand, or alternatively which is analogous to the ligand or is the ligand itself, where the second protein is bound to colored particles such as a metal sol or colloid, preferably gold.

Each of the at least two capture zones comprises at least a protein immobilized in the capture zone, the protein being complementary to the ligand. In some embodiments, the affinity to which the protein present in the first capture zone binds to the ligand differs from the affinity to which the protein present in the second capture zone binds the ligand.

The sample is contacted with the sample receiving zone, whereby the sample flows along the flow path. Quantitation is carried out by observing the pattern of label that accumulates at the one or more capture zones and correlating that pattern to the amount of ligand in the sample.

In one embodiment disclosed by U.S. Pat. No. 7,144,742, the first capture zone binds to and depletes some of the complex in the sample. Therefore, the concentration of complex which reaches the second capture zone is lower, having been depleted by the quantity of the complex which bound to the first capture zone, and the rate of binding of complex to the second capture zone is lower than the rate of binding of complex to the first capture zone. As such, for a given amount of ligand in the sample, a detectable signal takes longer to appear on the second capture zone relative to the first capture zone. This concept can be applied to a third sequential zone, a fourth, etc. For example, a low concentration of ligand may only produce signal on the two most upstream capture zones, a higher ligand concentration may produce signal on the three most upstream capture zones, an even higher ligand concentration will produce signal on the four most upstream capture zones, and so on. Therefore, the number of lines with detectable signal is proportional to the amount of ligand present in the sample.

Dipstick assays, as typified by home pregnancy and ovulation detection kits, are also a type of rapid test device. As described in U.S. Pat. No. 5,559,041, immunochemical components such as antibodies are bound to a solid phase. The assay device is “dipped” for incubation into a sample suspected of containing an antigen or ligand. Enzyme-labeled antibody is then added, either simultaneously or after an incubation period. The device next is washed and then inserted into a second solution containing a substrate for the enzyme. The enzyme-label, if present, interacts with the substrate, causing the formation of colored products which either deposit as a precipitate onto the solid phase or produce a visible color change in the substrate solution. Baxter et al., EP-A 0 125 118, disclose such a sandwich type dipstick immunoassay. Kali et al., EP-A 0 282 192, disclose a dipstick device for use in competition type assays.

There exists a need for a GL3 detection assay which is accurate, reliable and sufficiently sensitive to detect very low levels of GL3 that exist in body tissues and fluids. The immunoassays described above provide a means to sensitively and reliably detect protein antigens. However, the efficacy of these immunoassays is limited to the specificity, affinity and avidity of the antibodies to the antigen of interest.

As described above, antibodies against various glycolipids, and GL3 in particular, individually tend to have low binding affinities and/or are not specific for both the oligosaccharide and lipid moieties of GL3. The low affinities of most anti-carbohydrate antibodies appear to be related to rapid dissociation rates, MacKenzie et al., (1996). The Journal of Biological Chemistry, 271, 1527-1533. Mackenzie et al. also notes that antibodies in complex with carbohydrate antigens that have been determined at high-resolution show stacking interactions and hydrogen bonds formed between antigen and antibody were predominantly responsible for the interactions resulting in comparatively low affinity binding.

Further, GL3 exists as a mixture of structural iso forms containing acyl chains ranging from 16 to 24 carbons in length with various degrees of saturation and hydroxylation. Roddy, T. P. et al. (2005) Clinical Chemistry 51: 237-240. These variations make the generation of antibodies which specifically bind GL3 challenging.

Additionally, use of the “sandwich” type immunoassays described above, encompassing a complex comprising a first GL3 binding protein, GL3 and a second GL3 binding protein, requires a pair of GL3 binding proteins, preferably GL3 specific antibodies, each of which can specifically bind GL3 simultaneously. Due to the small size of GL3 (˜1000 D), steric hindrance generated by a first GL3 specific binding protein, e.g., antibody, binding to GL-3 may prevent binding by a second GL3 specific binding protein, e.g., antibody. Additionally, conformational changes in GL3 induced by the binding of a first GL3 specific protein, may also contribute to diminished binding by a second GL3 binding protein.

Analysis of the structure of the small sized GL3 molecules and their epitopes support the hypothesis that the number, density and geometrical arrangement of the epitopes on GL3 may profoundly affect the ability of GL3 to simultaneously bind two GL3 specific binding proteins or antibodies.

Thus, as an essential element of developing a GL3 detection assay, there is the need for identifying moieties that can specifically bind GL3, both singularly and in concert with at least one other GL3 binding moiety, despite the small size and high lipid component of GL3.

### BRIEF SUMMARY

The invention pertains to a sandwich immunoassay to detect GL3, utilizing a pair of peptides which Applicant has surprisingly discovered specifically bind GL3 simultaneously. In one embodiment, the pair of GL3 binding peptides includes the antibodies BGR23 and GTC-1A, described herein. In another embodiment, the pair is composed of two identical GL3 binding peptides with specificity to an epitope present at multiple sites on GL3.

Specific embodiments include assays for detecting GL3 in a sample by means of a traditional sandwich assay, where GL3 comprises a first and a second GL3 binding site, and where the assay includes the following steps;
(A) contacting GL3 in the sample with a first peptide that specifically binds GL3 at the first GL3 binding site, under conditions which provide for formation of a first complex comprising the first peptide and GL3,
(B) contacting said first complex with a second peptide that specifically binds GL3 at the second GL3 binding site, under conditions which provide for formation of a second complex comprising the first complex and the second peptide, and
(C) detecting said second complex,
   wherein detection of said second complex in step (C) is indicative of GL3's presence in said sample and/or where the amount of second complex detected in step (C) reflects the level of GL3 in said sample, and/or where the amount of second complex detected in step (C) is directly correlated with the level of GL3 in said sample.

In one embodiment of the above traditional sandwich assay, the second GL3 binding peptide is attached to a surface, including, but not limited to an ELISA plate, a dipstick, an Immuno™ Stick (Nunc A/S), a chip, and an immunostrip. In another embodiment of the above assay the first and/or the second peptide that specifically binds GL3 is an antibody, optionally a monoclonal antibody, or fragment thereof.

A further embodiment of the above traditional sandwich assay includes the additional step of quantitating the amount of GL3 in the sample, preferably using control samples containing known amounts of GL3. Including a quantitation step in the above assay provides for methods to detect and diagnose Fabry's disease in a patient suspected of being afflicted with Fabry's disease, including in asymptomatic patients. In one embodiment described herein there is a method of detecting Fabry's disease in a patient comprising assaying the level of GL3 in a urine or plasma sample of said patient, wherein said GL3 comprises a first and a second binding site, said method comprising;
(A) incubating said sample with a first GL3 binding peptide that specifically binds said first binding site, under conditions which provide for formation of a first complex comprising said first GL3 binding peptide and said GL3,
(B) contacting said first complex with a second GL3 binding peptide that specifically binds said second binding site, under conditions which provide for formation of a second complex comprising said first complex and said second GL3 binding peptide, and
(C) detecting and quantitating said second complex,
   wherein the amount of second complex detected in step (C) reflects the level of GL3 in said sample, and wherein a level of GL3 detected in said sample which is at least 2 fold higher than that of a healthy control is indicative of Fabry's disease.

Including a quantitation step in the traditional sandwich assay also provides for methods to monitor the efficacy of treatment of Fabry's disease. In one embodiment there is a method of monitoring the efficacy of therapeutic treatment of Fabry's disease in a patient, comprising assaying the level of GL3 in a urine or plasma sample of said patient, wherein said GL3 comprises a first and a second binding site, comprising;
(A) incubating said sample with a first GL3 binding peptide that specifically binds said first binding site, under conditions which provide for formation of a first complex comprising said first GL3 binding peptide and said GL3,
(B) contacting said first complex with a second GL3 binding peptide that specifically binds said second binding site, under conditions which provide for formation of a second complex comprising said first complex and said second GL3 binding peptide, and
(C) detecting and quantitating said second complex,
   wherein the amount of second complex detected in step (C) reflects the level of GL3 in said sample, and wherein a decrease in concentration GL3 in said sample relative to that in a previous sample of said patient indicates said treatment of Fabry's disease in said patient is efficacious.

### Inhibitory Assay

Other embodiments described herein include assays for detecting GL3 in a sample by means of an inhibitory sandwich assay, where GL3 comprises a first and a second GL3 binding site, where the assay includes the following steps;
(A) providing a first complex comprising GL3 bound to a first GL3 binding peptide immobilized on a surface, wherein said first GL3 binding peptide specifically binds said first binding site, comprising the steps of:
(B) incubating said sample with a second GL3 binding peptide, wherein said second GL3 binding peptide specifically binds said second binding site, under conditions which provide for formation of a second complex comprising said second binding peptide and GL3 from said sample and;
(C) incubating the components of step (B) with said first complex, under conditions which provide for the formation of a third complex comprising said first complex and said second GL3 binding peptide; and
(D) detecting said third complex, if present,
   wherein a lack of detectable said third complex in step (D) indicates the presence of GL3 in said sample, and/or wherein detection of said third complex in step (D) indicates a lack of detectable GL3 in said sample, and/or wherein the level of GL3 in said sample is inversely correlated to the level of said third complex detected in step (D).

Step (A) of the above inhibitory sandwich assay which provides a first complex comprising GL3 bound to a first GL3 binding peptide immobilized on a surface, wherein said first GL3 binding peptide specifically binds said first binding site, can be accomplished by many methods, including, but not limited to: (i) immobilizing said first GL3 binding peptide to said surface, and (ii) incubating said first GL3 binding peptide with GL3 under conditions which provide for formation of a first complex comprising said first GL3 binding peptide bound to GL3 at said first site.

In one embodiment of the above inhibitory sandwich assay, the first complex comprising GL3 and said first GL3 binding peptide is attached to a surface, including, but not limited to an ELISA plate, a dipstick, an Immuno™ Stick (Nunc A/S), a chip, and an immunostrip. In another embodiment of the above assay the first and/or the second peptide that specifically binds GL3 is an antibody, optionally a monoclonal antibody, or fragment thereof.

A further embodiment of the above inhibitory sandwich assay includes the additional step of quantitating the amount of GL3 in the sample, preferably using control samples containing known amounts of GL3. Including a quantitation step in the above assay provides for methods to detect and/or diagnose Fabry's disease in a patient suspected of being afflicted with Fabry's disease, even in asymptomatic patients. One embodiment of a method of detecting Fabry's disease in a patient comprising assaying the level of GL3 in a urine or plasma sample of said patient, wherein said GL3 comprises a first and a second GL3 binding site, comprises;
(A) providing a first complex comprising GL3 bound to a first GL3 binding peptide immobilized on a surface, wherein said first GL3 binding peptide specifically binds said first GL3 binding site, comprising the steps of:
(B) incubating said sample with a second GL3 binding peptide, wherein said second GL3 binding peptide specifically binds to said second binding site, under conditions which provide for formation of a second complex comprising said second binding peptide and GL3 from said sample and;
(C) incubating the components of step (B) with said first complex, under conditions which provide for the formation of a third complex comprising said first complex and said second GL3 binding peptide; and
(D) detecting and quantitating said third complex, if present,
   wherein a lack of detection of said third complex in step (D) indicates the presence of GL3 in said sample, wherein the concentration of GL3 in said sample is inversely correlated to the amount of said third complex detected, and wherein a level of GL3 detected in said sample which is at least 2 fold higher than that of a healthy control is indicative of Fabry's disease in said patient.

Step (A) of the above inhibitory sandwich assay which provides a first complex comprising GL3 bound to a first GL3 binding peptide immobilized on a surface, wherein said first GL3 binding peptide specifically binds the first GL3 binding site, can be accomplished by many methods, including, but not limited to: (i) immobilizing said first GL3 binding peptide to said surface, and (ii) incubating said first GL3 binding peptide with GL3 under conditions which provide for formation of a first complex comprising said first GL3 binding peptide bound to GL3 at said first site.

Including a quantitation step in the above assay also provides for methods to monitor the efficacy of treatment of Fabry's disease. One embodiment of a method of monitoring the efficacy of therapeutic treatment of Fabry's disease in a patient, comprises assaying the level of GL3 in a urine or plasma sample of said patient, wherein said GL3 comprises a first and a second GL3 binding site, comprising;
(A) providing a first complex comprising GL3 bound to a first GL3 binding peptide immobilized on a surface, wherein said first GL3 binding peptide specifically binds said first binding site, comprising the steps of:
(B) incubating said sample with a second GL3 binding peptide wherein said second GL3 binding peptide specifically binds to said second binding site, under conditions which provide for formation of a second complex comprising said second binding peptide and GL3 from said sample and;
(C) incubating the components of step (B) with said first complex, under conditions which provide for the formation of a third complex comprising said first complex and said second GL3 binding peptide; and
(D) detecting and quantitating said third complex, if present,
   wherein a lack of detection of said third complex in step (D) indicates the presence of GL3 in said sample, wherein the level of GL3 in said sample is inversely correlated to the amount of said third complex detected, and wherein a level of GL3 detected in said sample which is less than that of a previous sample of said patient indicates said treatment of Fabry's disease in said patient is efficacious.

Step (A) of the above inhibitory sandwich assay which provides a first complex comprising GL3 bound to a first GL3 binding peptide immobilized on a surface, wherein said first GL3 binding peptide specifically binds the first GL3 binding site, can be accomplished by many methods, including, but not limited to: (i) immobilizing said first GL3 binding peptide to said surface, and (ii) incubating said first GL3 binding peptide with GL3 under conditions which provide for formation of a first complex comprising said first GL3 binding peptide bound to GL3 at said first site.

Other embodiments described herein include assays for detecting GL3 in a sample by means of an inhibitory based assay where the assay includes the following steps;
(A) immobilizing GL3 to a surface,
(B) incubating said sample with a GL3 binding peptide under conditions which provide for formation of a complex comprising GL3 from said sample and said GL3 binding peptide,
(C) incubating the components of step (B) with the immobilized GL3 of step (A), under conditions which provide for the formation of a second complex comprising said immobilized GL3 of step (A) and said GL3 binding peptide, and
(D) detecting said second complex, if present,
   wherein a lack of detectable said third complex in step (D) indicates the presence of GL3 in said sample, and/or wherein detection of said third complex in step (D) indicates a lack of detectable GL3 in said sample, and/or wherein the level of GL3 in said sample is inversely correlated to the level of said third complex detected in step (D).

In one embodiment of the above inhibitory assay, the GL3 is attached to a surface, including, but not limited to an ELISA plate, a dipstick, an Immuno™ Stick (Nunc A/S), a chip, and an immunostrip. In another embodiment of the above assay the peptide that specifically binds GL3 is an antibody, optionally a monoclonal antibody, or fragment thereof.

A further embodiment of the above inhibitory assay includes the additional step of quantitating the amount of GL3 in the sample, preferably using control samples containing known amounts of GL3. Including a quantitation step in the above assay provides for methods to detect and/or diagnose Fabry's disease in a patient suspected of being afflicted with Fabry's disease, even in asymptomatic patients. In one embodiment a method of detecting Fabry's disease in a patient comprises assaying the level of GL3 in a urine or plasma sample of said patient comprising;
(A) immobilizing GL3 to a surface,
(B) incubating said sample with a GL3 binding peptide under conditions which provide for formation of a complex comprising GL3 from said sample and said GL3 binding peptide,
(C) incubating the components of step (B) with the immobilized GL3 of step (A), under conditions which provide for the formation of a second complex comprising said immobilized GL3 of step (A) and said GL3 binding peptide, and
(D) detecting said second complex, if present,
   wherein no detectable said second complex in step (D) indicates the presence of GL3 in said sample, wherein the level of GL3 in said sample is inversely related to the amount of said second compound detected in step (D), and wherein a 200 percent increase of GL3 in said sample relative to that in a healthy control is indicative of Fabry's disease.

Including a quantitation step in the above assay also provides for methods to monitor the efficacy of treatment of Fabry's disease. In one embodiment a method of monitoring the efficacy of therapeutic treatment of Fabry's disease in a patient, comprises assaying the level of GL3 in a urine or plasma sample of said patient, comprising;
(A) immobilizing GL3 to a surface,
(B) incubating said sample with a GL3 binding peptide under conditions which provide for formation of a complex comprising GL3 from said sample and said GL3 binding peptide,
(C) incubating the components of step (B) with the immobilized GL3 of step (A), under conditions which provide for the formation of a second complex comprising said immobilized GL3 of step (A) and said GL3 binding peptide, and
(D) detecting said second complex, if present
   wherein no detectable said second complex in step (D) indicates the presence of GL3 in said sample, wherein the level of GL3 in said sample is inversely related to the amount of said second compound detected in step (D), and wherein a level of GL3 detected in said sample which represents a decrease in concentration GL3 in said sample relative to that in a previous sample of said patient indicates said treatment of Fabry's disease in said patient is efficacious.

Other aspects of the invention are discussed infra, including kits useful in practicing the claimed invention and methods of screening pairs of GL3 ligand for their ability to simultaneously bind GL3, including assays in which at least one of the GL3 ligands is immobilized. Also described herein are methods of screening pairs of antibodies and or proteins which specifically bind GL3 simultaneously.

### DEFINITIONS

GL3 is a glycolipid known as globotriaosylceramide (also known as GL3, GB3, CTH, trihexosyl ceramide, ceramide trihexosamide). GL3 exists as a mixture of structural isoforms containing acyl chains ranging from 16 to 24 carbons in length with various degrees of saturation and hydroxylation, Roddy et al. Clinical Chemistry. 2005; 51:237-240, see FIG. 11. GL3 is hydrolyzed by the enzyme alpha galactosidase A. Diminished alpha galactosidase A activity results in progressive accumulation of GL3 in cells.

As used herein, a “GL3 ligand” is a molecule which specifically binds to GL3. Preferably, the GL3 ligand is a protein, polypeptide or peptide, or fragment thereof, which specifically binds to GL3. As used herein, a “GL3 binding peptide” is a protein, polypeptide or peptide, or fragment thereof, which specifically binds to GL3. In other embodiments the GL3 binding peptide is an antibody or fragment thereof, which specifically binds GL3. In other embodiments the GL3 binding peptide is protein, polypeptide or peptide, other than an antibody or fragment thereof, which specifically binds GL3. In other embodiments the GL3 binding peptide is a protein, polypeptide or peptide having an antigen-binding site of an antibody, or having the requisite CDR region(s) of a GL3 binding antibody.

As used herein, the term “antibody” means an immunoglobulin molecule or a fragment of an immunoglobulin molecule having the ability to specifically bind to a particular antigen. Antibodies are well known to those of ordinary skill in the science of immunology. As used herein, the term “antibody” means not only full-length antibody molecules but also fragments of antibody molecules retaining antigen binding ability. Such fragments are also well known in the art and are regularly employed both in vitro and in vivo. In particular, as used herein, the term “antibody” means not only full-length immunoglobulin molecules but also antigen binding active fragments such as the well-known active fragments F(ab′)₂, Fab, Fv, and Fd.

The antibody can be a human antibody, a chimeric antibody, a recombinant antibody, a humanized antibody, a monoclonal antibody, or a polyclonal antibody. The antibody can be an intact immunoglobulin, e.g., an IgA, IgG, IgE, IgD, IgM or subtypes thereof. The antibody can be conjugated to a functional moiety (e.g., a compound which has a biological or chemical function (which may be a second different polypeptide, a therapeutic drug, a cytotoxic agent, a detectable moiety, or a support. An antibody interacts with its epitope with high affinity and specificity, binding with an affinity constant of at least 10⁷ M⁻¹, preferably, at least 10⁸ M⁻¹, 10⁹ M⁻¹, or 10¹° M⁻¹.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986, supra; Roitt, 1991, supra). In both the heavy chain Fd fragment and the light chain of IgG immunoglobulins, there are four framework regions (FRI through FR4) separated respectively by three complementarity determining regions (CDR1 through CDR3). The CDRs, and in particular the CDR3 regions, and more particularly the heavy chain CDR3, are largely responsible for antibody specificity.

Antibodies useful in the invention may be made using a mammal, e.g. rat, hamster, rabbit, chicken, mouse or goat. The program for inoculation is not critical and may be any normally used for this purpose in the art. Such procedures are described, for example, in Antibodies A Laboratory Manual, Cold Spring Harbor Laboratory, 1988, pages 92-115.

### Polyclonal Antibodies

In one embodiment, the GL3 specific antibodies are polyclonal antibodies. Methods for preparing polyclonal antibodies are known to the skilled artisan. Polyclonal antibodies can be raised in an animal, for example, by one or more injections of GL3 and, if desired, an adjuvant. Typically, GL3 and/or adjuvant will be injected in the mammal by multiple subcutaneous or intraperitoneal injections. The preferred antibodies are highly sensitive for the detection of GL3. Highly sensitivity antibodies are useful for detection of low concentrations of GL3 in bodily samples, e.g. whole blood, blood plasma, and urine.

### Monoclonal Antibodies

The GL3 antibodies described herein are monoclonal antibodies. Monoclonal antibodies can be prepared using hybridoma methods, such as those described by Kohler and Milstein, Nature, 256:495 (1975). In a hybridoma method, a mouse, hamster, or other appropriate host animal, is typically immunized with GL3 to elicit lymphocytes that produce or are capable of producing antibodies that will specifically bind to GL3. Alternatively, the lymphocytes may be immunized in vitro.

As used herein, the phrase “specifically binds to” refers to an antibody, reagent or binding moiety's binding of a ligand with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art (for example, by Scatchard analysis and surface plasma resonance). A variety of immunoassay formats can be used to select antibodies specifically immunoreactive with a particular antigen. For example, solid-phase ELISA immunoassays are routinely used to select monoclonal antibodies specifically immunoreactive with a ligand. See Harlow and Lane, Antibodies: A Laboratory Manual, Cold Springs Harbor Publications, New York, (1988) for a description of immunoassay formats and conditions that can be used to determine specific immunoreactivity. Typically, a specific or selective reaction will be at least twice background signal to noise and more typically, more than 10 to 100 times greater than background.

The term “sample” as used herein refers to any material, including any biological or organic material that could contain GL3 for detection. Preferably the biological sample is in liquid form or can be changed into a liquid form. Preferably, the sample comprises a bodily fluid such as blood, blood plasma, urine, etc.

As used herein, the terms “immobile” or “immobilized” or “irreversibly bound” refer to reagents such as GL3 binding peptides and GL3 itself, which are attached to a membrane, substrate or other support, such that contact with the liquid sample, or lateral flow or capillary flow of the liquid sample, does not alter the location of the immobilized reagent in or on the support. For example, in the sandwich assays, once the immobilized GL3 binding peptide forms a complex with GL3 and another GL3 binding peptide which is labeled, e.g., bound to colored particulate label, the complex is prevented from continuing with the flow of the liquid sample. Such an attachment of the immobilized GL3 binding peptide can be through e.g., covalent, ionic or hydrophobic means. Thus, in the immobilization of GL3 and GL3 binding peptides or antibodies, physical adsorption may be used. Alternatively, chemical binding that is conventionally used for immobilization of proteins, enzymes, etc. may be used as well. Those skilled in the art will be aware of methods available for attachment to immobilize various reagents.

As used herein, the phrase “irreversibly bound”, and the terms “immobile” or “immobilized” refer to reagents which are attached to a membrane, substrate or support such that flow, including lateral flow or capillary flow, of the liquid, including liquid sample, does not alter the location of the immobile reagent in or on the support. Such attachment can, e.g., be through covalent, ionic or hydrophobic means. Those skilled in the art will be aware of methods available for attachment to immobilize various reagents.

As used herein, the phrase “reversibly bound” refers to reagents which are attached to a membrane, substrate or support, such that flow, including lateral flow or capillary flow, of the liquid, including liquid sample, upon contact with the reversibly bound reagent, releases the reversibly bound reagent from the membrane, substrate or support to which the reagent was attached.

As used herein, “GL3” is ceramide trihexosamide, and “lyso-GL3” is lyso-ceramide trihexosamide.

As used herein, the phrase “site on GL3”, in the context of a GL3 binding peptide binding GL3, is referred to as the ligand binding site on GL3 that an individual GL3 binding peptide specifically binds. When the GL3 binding peptide is an antibody, or an antibody fragment or derivative, the ligand binding site is the epitope on GL3 to which the GL3 antibody specifically binds. A first binding site on GL3 and a second binding site on GL3 can contain different epitopes, or alternatively a first binding site on GL3 and a second binding site on GL3 can contain the same epitope located at two distinct sites of GL3.

As used herein, the phrase “site on Lyso-GL3” to which a Lyso-GL3 binding peptide binds Lyso-GL3 is referred to as the ligand binding site on Lyso-GL3 that an individual Lyso-GL3 binding peptide specifically binds. When the Lyso-GL3 binding peptide is an antibody, or an antibody fragment or derivative, the ligand binding site is the epitope on Lyso-GL3 to which the GL3 antibody specifically binds.

As used herein, the terms “Contacting” or “Incubating” includes the step of reacting a sample being analyzed for its GL3 content with a GL3 binding peptide or antibody for a sufficient amount of time under conditions that promote the binding to GL3 if present in the sample, by a GL3 binding peptide or antibody. It will be understood by those skilled in the art that the immunoassay reagents and sample may be reacted in various conditions to achieve this step.

“Complexes” as used herein refer to the combination products formed as a result of these reactions of the assays described herein and referenced above. As such, these products include, but are not limited to, products which comprise, for example, a GL3-GL3 binding peptide formation, or a “sandwich” which comprises a second GL3 binding peptide bound to the GL3-GL3 binding peptide formation. Thus the term “complex” includes any heterogeneous or homogeneous, sandwich formation produced for or during an assay for the detection of GL3 in a sample described herein.

In some embodiments of the assays described herein, a physical means is employed to separate complexes bound to the solid phase from unbound reagents such as filtration of particles, decantation of reaction solutions from coated tubes or wells, magnetic separation, capillary action, and other means known to those skilled in the art. It will also be understood that a separate washing of the solid phase may be included in the assays described herein.

The resulting reaction mixture, or complexes, are prepared and/or formed in a solution that optimizes the binding of GL3 by the GL3 binding peptides. An appropriate solution is an aqueous solution or buffer. The solution is preferably provided under conditions that will promote specific binding, minimize non-specific binding, stabilize and preserve reagent reactivity, and may contain buffers, detergents, solvents, salts, chelators, proteins, polymers, carbohydrates, sugars, and other substances known to those skilled in the art.

The contacting or incubating steps of the assays described herein provide sufficient amount of time to allow the GL3 binding peptide to react and bind to the GL3 to form a GL3 binding peptide-GL3 complex or a GL-3 sandwich complex as described above. The shortest amount of reaction time that results in binding is desired to minimize the time required to complete the assay. An appropriate reaction time period for an immunostrip test is less than or equal to 10 minutes or between approximately one minute and 10 minutes. A reaction time of less than five minutes is preferred. Most preferably, the reaction time is less than three minutes. By optimizing the reagents, binding may be substantially completed as the reagents are combined.

The reaction is performed at any temperature at which the reagents do not degrade or become inactivated. A temperature between approximately 18° C. and 30° C. is preferred, including ambient or room temperature (approximately 22°).

The term “detection” as used with respect to the method steps of the assays described herein, provides for the identification of labeled molecules, by detection methods readily available to one of skill in the art.

To detect and quantitate GL3 in the assays described herein, the GL3 specific antibodies are labeled. “Direct Labeling” refers to the process of providing labels that are attached without an intermediary to a substrate, e.g., GL3 binding peptides or antibodies. “Indirect Labeling” refers to the process of providing labels that are attached with an intermediary to a substrate, for example, by reaction with labeled substances that bind to the antibody such as secondary antibodies, protein A or protein G.

As used herein, the term “label” includes a detectable indicator, including but not limited to labels which are soluble or particulate, metallic, organic, or inorganic, and may include spectral labels such as green fluorescent protein, fluorescent dyes (e.g., cyanine fluorescent dyes (e.g., Cy2, Cy3, Cy5, Cy5.5, Cy7 (manufactured by Amersham Biosciences) fluorescein and its derivatives, fluorescamine, fluorescein isothiocyanate, etc., rhodamine) chemiluminescent compounds (e.g., luciferin and luminol), enzymes (e.g., β-galactosidase, β-glucosidase, alkaline phosphatase, peroxidase, malate dehydrogenase, etc.), radioisotopes (e.g., [¹²⁵I], [¹³¹I], [³H], [¹⁴C], [³²P], [³³P], [³⁵S], etc.), luminescent substances (e.g., luminol, a luminol derivative, luciferin, lucigenin, etc.), biotin, lanthanides, etc. a biotin-avidin system may be used as well for binding an antibody to a labeling agent, spectral calorimetric labels such as colloidal gold, or carbon particles, or colored glass or plastic (e.g. polystyrene, polypropylene, latex, etc.) beads. Where necessary or desirable, particle labels can be colored, e.g., by applying dye to particles.

As used herein, the term “colored particle label” includes, but is not limited to, colored latex (polystyrene) particles, metallic (e.g. gold) sols, non-metallic elemental (e.g. Selenium, Carbon) sols and dye sols. In one embodiment, a colored particle label is a colored particle that further comprises a member of a conjugate pair. Examples of colored particles that may be used include, but are not limited to, organic polymer latex particles, such as polystyrene latex beads, colloidal gold particles, colloidal sulphur particles, colloidal selenium particles, colloidal barium sulfate particles, colloidal iron sulfate particles, metal iodate particles, silver halide particles, silica particles, colloidal metal (hydrous) oxide particles, colloidal metal sulfide particles, carbon black particles, colloidal lead selenide particles, colloidal cadmium selenide particles, colloidal metal phosphate particles, colloidal metal ferrite particles, any of the above-mentioned colloidal particles coated with organic or inorganic layers, protein or peptide molecules, or liposomes.

As used herein, the term “quantitating” refers to the means used to determine the concentration of GL3 in a sample. In some embodiments of the assays described herein, the concentration of GL3 in the sample is determined by comparing the intensity of the color produced by the sample to a color card, by using a reflectometer, or by using a spectrophotometer or microtiter plate reader.

As used herein, the term “Surface” describes a carrier to which GL3 binding peptides, and in some instances exogenous GL3 can be attached. The GL3 binding peptides can be bound to many different surfaces and used to detect the presence of GL3. Examples of well-known surfaces include glass, synthetic resins such as polyacrylamide, silicone, polystyrene, polypropylene, polyethylene, dextran, nylon, amylase, natural and modified cellulose, polyacrylamide, insoluble polysaccharides such as agarose, and magnetite. The nature of the surfaces can be either soluble or insoluble for purposes of the invention. Those skilled in the art will know of other suitable surfaces for GL3 binding peptides, or will be able to ascertain such, using routine experimentation. In various embodiments of the assays described herein, surfaces include membrane surfaces, Immuno™ Stick surfaces and ELISA plate surfaces. An Immuno™ Stick apparatus, or dipstick, or the like, comprises (i) a tube manufactured with low protein binding material, e.g., polypropylene, in which the sample of interest and other reagents can be held or incubated in, and (ii) a synthetic resin carrier, e.g., polystyrene shaped paddles or other shape, e.g., plates, spheres, reagent tubes, strips and rodlets, which can be uniformly coated with immunologically-active material, e.g., with an antibody specific for GL3 or with an antibody specific for GL3 complexed with GL3, similar to a microtiter well of an ELISA plate.

In some embodiments of the assays described herein, a sample is analyzed by means of a biochip. As used herein, a “biochip” comprises solid substrates with a generally planar surface, to which a capture reagent is attached. In some embodiments, the surface of a biochip comprises a plurality of addressable locations, each of which has the capture reagent bound there.

Protein biochips are biochips adapted for the capture of polypeptides. Many protein biochips are described in the art. These include, for example, protein biochips produced by Ciphergen Biosystems, Inc. (Fremont, Calif.), Zyomyx (Hayward, Calif.), Invitrogen (Carlsbad, Calif.), Biacore (Uppsala, Sweden) and Procognia (Berkshire, UK). Examples of such protein biochips are described in the following patents or published patent applications: U.S. Pat. No. 6,225,047 (Hutchens & Yip); U.S. Pat. No. 6,537,749 (Kuimelis and Wagner); U.S. Pat. No. 6,329,209 (Wagner et al.); PCT International Publication No. WO 00/56934 (Englert et al.); PCT International Publication No. WO 03/048768 (Boutell et al.); U.S. Pat. No. 6,902,897 (Tweedie et al.) and U.S. Pat. No. 5,242,828 (Bergstrom et al.).

Other features and advantages of the invention will be apparent from the following detailed description of the invention in conjunction with the accompanying drawings and from the claims.

### BRIEF DESCRIPTION OF THE DRAWINGS

FIG. 1 illustrates an outline of the assay of an ELISA based upon an IgG (monoclonal) coating antibody and an IgM (monoclonal) detector Ab used to detect GL3 or Lyso-GL3 in a sample.

FIG. 2 presents data indicating that a population of Fabry samples can be distinguished from normal samples using the GL3 ELISA assay illustrated in FIG. 1.

FIG. 3 presents data from a semiquantitative assay based on the concept of the ELISA assay illustrated in FIG. 1. The data show an increase in color of the paddles with increasing amounts of GL3 in the samples.

FIG. 4 illustrates a comparison between the steps of the traditional ELISA based assay described in FIG. 1 and a sensitive inhibitor assay for detecting GL3 or Lyso-GL3.

FIG. 5 displays the results of a GL3 inhibition Lateral Flow assay designed to test the limits of this assay in detecting GL3 detection in a buffer matrix.

FIG. 6 illustrates how a Biacore assay can be used to determine which pairs of GL3 binding peptides can be used to detect GL3 in a sandwich assay. The schematic in the left panel of FIG. 6 diagrams the two steps; in the first step a liquid sample containing GL3 is added to an immobilized first GL3 binding peptide (antibody), in the second step a liquid solution containing a second GL3 binding peptide (antibody) is added. If the second GL3 binding peptide binds an accessible site on GL3 bound to the immobilized first GL3 binding peptide, then a sandwich assay is formed. If the second GL3 binding peptide does not bind an accessible site on GL3 bound to the immobilized first GL3 binding peptide, (as in the case where the first and second GL3 binding peptides bind the same epitope), then no sandwich assay is formed. The left panel of FIG. 6 illustrates the readout from the Biacore assay over time. The graph illustrates an increase in resonance units with the binding of GL3, followed by a further increase in resonance units if a sandwich is formed. If no sandwich is formed, then there is no further increase in resonance units.

FIG. 7 displays data from a Biacore assay designed to screen for peptides capable of binding to GL3 bound to an immobilized first GL3 binding peptide. The data show clear binding of 500 nM GTC-1A (top curve) to GL3 bound to immobilized BGR23. These results suggest that the antibodies GTC-1A and BGR23 bind to different epitopes on GL3.

FIG. 8 displays data from a Biacore assay designed to screen for peptides capable of binding to Lyso GL3. Lyso GL3 showed low affinity to immobilized BGR23. The data show Lyso-GL3 displayed very weak binding to the remaining immobilized antibodies (GTC-1A and 38-13) and to immobilized beta subunit of Verotoxin.

FIG. 9 displays data from a Biacore assay designed to screen for peptides capable of binding to GL3 bound to an immobilized first GL3 binding peptide. The data show that Verotoxin beta subunit (VTB) displayed clear binding to 5 μM GL3 captured by immobilized GL3 binding peptide antibody BGR23 in a Biacore Assay. This GL3 sandwich containing VTB and BGR23 suggests that VTB binds a different binding epitope on GL3 from BGR23.

FIG. 10 displays data from a Biacore assay designed to screen for peptides capable of binding to Lyso-GL3 bound to an immobilized first Lyso-GL3 binding peptide. The data show that Verotoxin beta subunit (VTB) displayed binding to 5 μM Lyso-GL3 captured by immobilized BGR23 in a Biacore Assay. This Lyso-GL3 sandwich containing VTB and BGR23 suggests that VTB binds a different binding epitope on Lyso-GL3 from BGR23.

FIG. 11 displays the structure of GL3 iso forms.

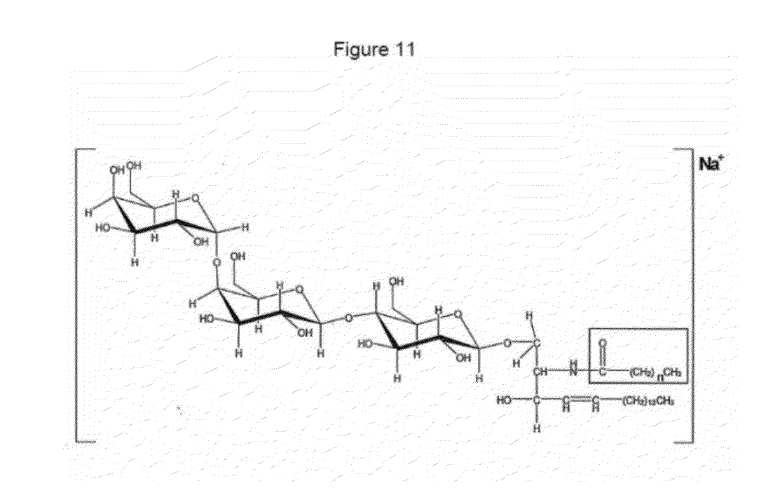

### DETAILED DESCRIPTION OF THE INVENTION

The invention pertains to a sandwich immunoassay to detect GL3, utilizing a pair of GL3 binding peptides which bind GL3 simultaneously. Specifically, Applicant has developed a sandwich based immunoassay which utilizes a pair of GL3 binding peptides, e.g., GL3 specific monoclonal antibodies, one for capture and one for detection, to create an antibody “sandwich” around GL3. The capture antibody is optionally immobilized on a fixed surface, while the labeled GL3 antibody is added to the device either simultaneously with the addition of the sample or subsequent to the addition of the sample to the device. This immunoassay can be applied to several different formats with fixed surfaces, including Biacore assays, ELISAs and lateral flow assays, flow cytometry assays using microspheres, for example Luminex xMAP® microspheres readable using a series of lasers, the first of which determines the identity of the microsphere and the second the amount of bound reporter.

To further increase sensitivity, Applicant has modified these traditional sandwich based assays to provide an inhibition based assay by complexing the capture GL3 antibody, which is preferably immobilized, with GL3 before adding the sample and a second GL3 antibody which is labeled. In this inhibition based assay, any GL3 in the sample is complexed with the labeled antibody which in turn is inhibited from binding the immobilized capture antibody/GL3 complex. However, if the sample contains little or no GL3, the labeled antibody will bind the GL3 in the immobilized capture complex in a sandwich format. Therefore, the amount of GL3 in the sample is inversely correlated with the amount of labeled GL3 antibody binding to the immobilized capture antibody-GL3 complex.

In another embodiment of an inhibition based assay, the Capture antibody is replaced with immobilized GL3, termed “Capture GL3”, before adding sample or label antibody. In this inhibition based assay, any GL3 in the sample is complexed with the detector antibody, which is inhibited from binding the “Capture GL3”. However, if the sample contains little or no GL3, the labeled antibody will bind the immobilized “capture GL3”.

These assays can be applied to methods of diagnosing Fabry's disease, and to methods of monitoring the progression of disease in individuals afflicted with Fabry's disease, for example to monitor the effectiveness of therapy. These assays can also be applied to methods of monitoring the progression of disease in female carriers with Fabry disease and in those afflicted with α-galactosidase A deficiency. Also provided are kits comprising reagents for use in such a sandwich based assay. In the kits and methods described herein, the above mentioned antibodies directed to GL3 can be substituted with GL3 binding proteins. An example of an GL3 binding peptide includes the beta unit of E. coli Verotoxin.

Applicant has also applied the above mentioned methods and kits to the detection of a side product of GL3 called lyso-GL3. Lyso-GL3 is also known in the art to be dramatically elevated in plasma and urine of Fabry patients.

Fabry Disease is a recessive, X-linked inherited recessive lysosomal storage disease, caused by a deficiency in the lysosomal enzyme alpha-galactosidase A. Absence of this lysosomal hydrolase results in progressive deposition of the glycosphingolipid globotriasylceramide (GL3) in several tissues and fluids of the body including the vascular endothelium. Progressive endothelial accumulation of GL3, leads to ischemia and infarction in organs such as kidney, heart or brain, causing excruciating pain, kidney failure, cardiac and cerebrovascular disease. The average age of death for an affected individual, from renal, cardiac and/or cerebral complications of the vascular disease, is 41 years. (See, e.g., Desnick et al., in Scriver et al., eds. The Molecular Basis of Inherited Disease, 7^{th} Ed., Chapter 89, pp. 2741-2784, McGraw Hill (1995)). Methods for quantitating individual glycosphingolipids, such as TLC, TLC immunoblotting, TLC immunostaining, HPLC, and GL3, are either not sensitive enough or are too laborious and time-consuming to be of practical value for the rapid and high-throughput determinations of GL3 required for routine diagnostic studies and for monitoring efforts to treat Fabry's disease.

At the time of the invention most antiglycolipid antibodies are of the IgM subtype and therefore of low affinity. Also, they often recognize only the carbohydrate moieties and therefore may cross-react with other molecules. See Zeidner et al. 1999 Analytical Biochemistry 267:104-113.

However, despite the small size and lipid nature of GL3, Applicant was able to develop an assay using a pair of GL3 binding peptides, each binding simultaneously to GL3. In one embodiment, each of a pair of GL3 binding peptides binds to a different epitope or site on the GL3 molecule. In another embodiment, each of a pair of GL3 binding peptides binds to the same epitope or site on the GL3 molecule. In one embodiment, one member of the pair of GL3 binding peptides acts as the capture antibody and the second member of the pair GL3 binding peptides functions as the detection molecule.

The GL3 binding peptide pairs can be used in the in vitro assays described herein to diagnose and monitor the course of disease, in particular Fabry's disease, resulting from deficient galactosidase A function. Thus, for example, by measuring the increase or decrease in the amount of GL3 in various body fluids, in particular blood, blood plasma, and urine, a particular therapeutic regimen aimed at ameliorating the Fabry's disease can be monitored for its effectiveness in treating Fabry's disease.

Therefore, in one embodiment, an ELISA method was developed for the sensitive, reliable, and high-throughput quantitation of GL3 using a sandwich assay comprising the GL3 binding peptide pairs and GL3. In another embodiment the ELISA method was modified by replacing an ELISA plate with one or more dipsticks or Immuno™ Sticks (Nunc A/S). In another embodiment the sandwich assay was modified to produce an inhibitory assay, able to detect a concentration of GL3 in a sample at least as low as 125 ng/ml.

Globotriaosylceramide (GL3) is Galα1-4Galβ1-4Glc-Cer. Thus, globotriaosylceramide is formed of three sugars and a fatty substance called ceramide, and is found in most cells of the body. Normally globotriaosylceramide is metabolized to lactosylceramide by the enzyme alpha-galactosidase A. In patients with Fabry's disease, this enzyme does not function properly or is absent, and globotriaosylceramide cannot be broken down in cells, leading to its progressive accumulation. See the URL at nysbg.org/genetics/fabry/index.shtml.

Plasma levels of GL3 from classic hemizygotes (XY) range from 4.3 to 27.6 ug/ml GL3, while plasma levels of GL3 from heterozygotes (XY) range from 4.4-12.0 ug/ml GL3, compared to plasma levels of GL3 from healthy controls which range from 3.6-7.5 ug/ml GL3, Winchester et al. citing Mills et al. J. Inherit. Metabolic Dis. (2005) 28:35-48; Mills et al. Eur. J. Pediatr. 2004; 163:595-603; and Young et al. Acta Pediatr Suppl. (2005) 447-51-4. Winchester et al. also report urinary levels of GL3 from classic hemizygotes range from 0.12-2.80 mg GL3/mmol Creatinine (CR), while urinary levels from heterozygotes range from 0.02-0.37 mg GL3/mmol Cr, compared to urinary levels from healthy controls which range from 0.01-0.03 mg GL3/mmol Cr. Thus, an increase of at least 200% in GL3 in plasma or urine relative to normal healthy controls is indicative of Fabry's disease.

### GL3 Binding Molecules—Antibodies

BGR23 antibody binds GL3. It can be obtained from Seikagaku BioBusiness Corporation (product code# is 370680-8), and is also available through Cape Cod Associates-CATALOG #370680-1. The BGR23 antibody isotype is IgG2b and is produced by a mouse-hybridoma resulting from a fusion between PAI mouse myeloma cells and spleen cells from a C3H/Hen mouse immunized with purified glycolipid adsorbed to Salmonella minnesota, (Kotani, M., et al.: Arch. Biochem. Biophys., 310, 89-96 (1994)). According to Kotani, the globo-series glycolipids Gb3Cer, were used for immunization. None of the other various glycolipids or gangliosides tested were recognized by the BGR23 antibody.

GTC-1A antibody binds GL3 and is an IgM isotype produced by a mouse hybridoma cell line and can be accessed from Dr. Jan-Eric Mansson.

38-13 IgM, a monoclonal antibody, directed against a Burkitt lymphoma associated antigen has been described by Wiels, J., Fellous, M. and Tursz, T. Proc. Natl. Acad. Sci. USA. 78: 6485-6488.1981. 38.13 antibody was obtained by fusing murine myeloma cells with Lewis rat splenocytes sensitized with Daudi cells (human Burkitt lymphoma containing Epstein—Barr virus genome but lacking HLA-A, -B, and -C and beta 2-microglobulin molecules at the cell surface). 38.13 antibody was demonstrated to be a rat IgM.

### GL3 Binding Molecules—Non-Antibody Molecules

### Verotoxin

In an alternate embodiment, one antibody of the pair can be substituted for by the beta subunit of Escherichia coli verotoxin (VTB) in the assays described herein to determine the GL3 concentrations in urine, plasma and tissues, in particular from affected males and female carriers with Fabry disease and/or individuals with α-galactosidase A deficiency. The beta subunit of Escherichia coli verotoxin has been shown to have high specificity and avidity for GL3. Zeidner et al. Analytical Biochemistry (1999):267:104-113.

### GL3 Binding Peptides

The GL3 binding peptides encompassed by the methods and kits described herein are not limited to the specific antibodies and proteins listed above, i.e. BGR23 antibody, GTC-1A antibody, 38-13 antibody and the beta subunit of Verotoxin. The GL3 binding peptides encompassed by the methods and kits described herein also include proteins and molecules which specifically bind at the same site as at least one of BGR23 antibody, GTC-1A antibody, 38-13 antibody and Verotoxin. In another embodiment, the GL3 binding peptides encompassed by the methods and kits described herein also include proteins and molecules which compete with and/or inhibit the binding of at least one of BGR23 antibody, GTC-1A antibody, 38-13 antibody and Verotoxin from specifically binding GL3. In another embodiment, they encompass pairs of GL3 binding peptides which can bind GL3 and/or Lyso GL3 simultaneously. Further, in some embodiments they encompass pairs of GL3 binding peptides which can bind GL3 and/or Lyso GL3 simultaneously when GL3 and/or Lyso GL3 is bound to a surface.
Table 1 illustrates GL3 binding molecules useful in the instant inventions

| Antibody | Isotype | Binding Epitope | MW (Da) |
|---|---|---|---|
| 38.13 | Rat IgM | ? | ~900,000 |
| BGR23 | Mouse IgG2b | Gal α1 -- 4Galβ1 --4Glc-Cer | ~150,000 |
| GTC-1A | Mouse IgM | GalNAcβ1-4(NeuAcα2-3)Gal | ~900,000 |
| VTB | | ? | 7,700 |

### Lyso-GL3

Aerts et al. (PNAS Feb. 26, 2008 vol. 105 no. 8, pages 2812-2817) provides that although GL3 accumulation is clearly a prerequisite for manifestation of Fabry disease, these observations point to the existence of another factor in addition to GL3 that is involved in the pathogenesis of the disorder. Aerts et al. reported that plasma of Fabry patients contains markedly increased concentrations of deacylated GL3, globotriaosylsphingosine (lyso-GL3), noting that the relative increase in the plasma concentrations of this cationic amphiphilic glycolipid exceeds that of GL3 by more than an order of magnitude. Thus, an increase of more than ten fold the amount of lyso-GL3 found in urinary or plasma samples relative to healthy controls is indicative of Fabry's Disease. In another embodiment, the increase of up to and including 200%, 300%, 400%, 500%, 600%, 700%, 800%, 900% or 1000% the amount of lyso-GL3 found in urinary or plasma samples relative to healthy controls is indicative of Fabry's Disease. Aerts et al. reports that in contrast to GL3, Lyso-GL3 is a soluble compound that can easily move in and out cells. Lyso-GL3 lacks a hydrophobic (acyl) fragment compared to GL3. Lyso-GL3 is virtually not detectable in plasma obtained from normal individuals, but relatively high concentrations occur in samples from Fabry males. Also in the case of symptomatic Fabry females, increased levels of lyso-GL3 were detected. Compared to GL3, the abnormalities in plasma lyso-GL3 levels are far more pronounced in Fabry patients.

Similarly, WO2008075959 discloses that lyso-ceramide trihexosamide (lyso-GL3) is dramatically elevated in plasma of Fabry patients. WO2008075959 discloses that Lyso-GL3 is formed as a side-product from ceramide trihexosamide (GL3), either by ceramidase or protease activity. With the finding of aberrant plasma-levels of lyso-GL3 in Fabry patients, a unique tool for diagnosing and monitoring the treatment of Fabry disease is provided.

WO2008075959 discloses the laborious detection of lyso-GL3: (a) Bligh and Dyer extraction preferably followed by butanol/water extraction, optionally derivatized with a label and analyzed with a HPLC system preferably equipped with a reversed phase column, and (b) HPLC-tandem MS Bielawski et al in Methods. 2006 June; 39(2):82-91.

Using liquid chromatography-tandem mass spectrometry, Auray-Blais et al. 2008 March; 93(3):331-40. Epub 2007 Nov. 26, disclose that urinary Lyso-GL3 is a useful marker to evaluate Fabry disease, including evaluations that assessed the role of gender of Fabry patients and the role of treatment. Auray-Blais et al. found undetectable urinary levels of Lyso-GL3 in healthy controls. See Auray-Blais et al. 2008 March; 93(3):331-40. Epub 2007 Nov. 26, and unpublished results.

In order to provide a means for faster assays amenable to high throughput analysis, assays designed for the detection of Lyso GL3 using peptides that bind Lyso-GL3 are described herein. Further, the assays, methods and kits used for detection of GL3 described herein can be modified for use in detection of Lyso-GL3. For instance Lyso-GL3 binding peptides can be substituted for GL3 binding peptides. In some cases GL3 binding peptides can be used for the detection of Lyso-GL3. Applicant has found that many of the GL3 binding peptides described herein are capable of detecting Lyso-GL3.

### Sandwich Assays

In one embodiment, the focal point of methods and kits for detecting GL3 is the GL3 peptide binding sandwich or the GL3 antibody sandwich. In a GL3 peptide binding sandwich, a pair of GL3 binding peptides specifically binds GL3 at the same time, despite the small size of GL3 and its lipid components. A GL3 peptide sandwich can be detected in numerous assay formats, including, but not limited to, using a solid support which is an ELISA plate in an ELISA assay, a dipstick, an Immuno™ Stick (Nunc A/S) in a modified ELISA, a membrane like material in a lateral flow assay, or a protein chip in a BIAcore assay, for example.

In a traditional quantitative sandwich assay, there are three basic parts. For example, in such an assay for GL3, the GL3 in a sample, such as urine, is indirectly captured onto a solid phase such as an ELISA plate, dipstick or an Immuno™ Stick (Nunc A/S) using an immobilized GL3 binding peptide such as a primary antibody. In one embodiment, the primary antibody is the BGR32 monoclonal antibody. Then a “sandwich” is formed between the primary antibody or GL3 binding peptide, the GL3, and a second GL3 binding peptide such as a secondary antibody which is labeled and which has also been added to the incubation. In one embodiment, the labeled secondary antibody is the GTC-1A antibody. After a wash step, where unbound secondary antibody has been removed, the bound secondary antibody is detected, and quantitated against control samples containing known amounts of GL3.

Therefore, traditional quantitative immunoassay for the detection of GL3 comprises the steps of: a) providing a sample; b) incubating a portion of the sample with a primary anti-GL3 antibody which binds to the GL3, the primary antibody being bound to a solid carrier, and adding a secondary labeled antibody which binds to the GL3 to create an “antibody-GL3-antibody” complex, c) washing the antibody-GL3-antibody complex to remove unbound secondary antibody; d) measuring the amount of bound labeled antibody to determine the concentration of the GL3.

One advantage of lateral flow assays over other immunoassays is that the migration of the liquid sample and buffer along the flow path of the lateral flow assay obviates the need for washing GL3 compounds comprising one or more GL3 binding peptides, e.g., antibodies. As is known in the art, in a lateral flow device the sample, or an extract or dilution of said sample, which comprises the ligand(s) of interest, is permitted to flow laterally from the point of its application through one or more regions or zones of one or more membrane surfaces to a detection zone. The presence of the ligand in the applied sample can be detected by a variety of protocols, including direct visualization of visible moieties associated with the captured ligand. A lateral flow device comprises a material capable of transporting a solution by capillary action, i.e., wicking Different areas or zones in the strip contain the reagents needed to produce a detectable signal as the ligand is transported to or through such zones. The diffusional migration of the sample along the flow path provides an intrinsic washing after each GL3 complex is formed, whether the product was formed at a site along the porous membrane where the GL3 binding peptide is reversibly linked or at a site where the GL3 binding peptide is irreversibly linked.

Enzyme Linked Immunosorbent Assay (ELISA) methods that are used are based on the enzyme-linked immunosorbent assay (ELISA) techniques, require several steps of washing as the complexes of ligand and ligand specific proteins/antibodies are formed, and are described in, for example, Harlow, E., Lane D., Antibodies: a Laboratory Manual. 1998. Cold Spring Harbor Laboratory. pp 553-612. The ELISA method used in the present invention is described in Example 1.

For further details of such immunoassays, reference may be made to a variety of reviews or reference books, Eiji Ishikawa, et al. (ed.): “Enzyme Immunoassay” (published by Igaku Shoin, 1978); Eiji Ishikawa, et al. (ed.): “Enzyme Immunoassay” (Second Edition) (published by Igaku Shoin, 1982); Eiji Ishikawa, et al. (ed.): “Enzyme Immunoassay” (Third Edition) (published by Igaku Shoin, 1987); “Methods in Enzymology” Vol. 70 (Immunochemical Techniques (Part A)); ibid., Vol. 73 (Immunochemical Techniques (Part B)); ibid., Vol. 74 (Immunochemical Techniques (Part C)); ibid., Vol. 84 (Immunochemical Techniques (Part D: Selected Immunoassays)); ibid., Vol. 92 (Immunochemical Techniques (Part E: Monoclonal Antibodies and General Immunoassay Methods)); ibid., Vol. 121 (Immunochemical Techniques (Part I: Hybridoma Technology and Monoclonal Antibodies)) (published by Academic Press).

### Screening for GL3 Binding Peptide Pairs

A pivotal aspect to the sandwich assays for detecting GL3 described herein is obtaining a pair of GL3 peptide binding proteins that simultaneously bind GL3, despite the small size and lipid nature of GL3. As described above, Applicant has unexpectedly discovered pairs of GL3 binding peptides (e.g., BGR32 antibody/GTC-1A antibody and BGR32 antibody/Verotoxin beta subunit) that specifically bind GL3 at the same time for use in the sandwich assays for detecting GL3 as described herein. Pairs of GL3 binding peptides can be screened for their ability to bind GL3 simultaneously in a sandwich format using a BIAcore assay.

BIAcore technology provides for studying biospecific interactions in real time, without labeling any of the interactants (e.g., BIAcore). Changes in the optical phenomenon of surface plasmon resonance (SPR) can be used as an indication of real-time reactions between biological molecules. Biomolecular Interaction Analysis (BIA). Sjolander, S, and Urbaniczky, C. (1991) Anal. Chem. 63:2338-2345 and Szabo et al. (1995) Curr. Opin. Struct. Biol. 5:699-705. The peptide compound being tested can be purified peptide or can be operatively linked to a heterologous peptide or phage. That is, surface plasmon resonance can be used to ascertain if each of a pair of GL3 binding peptides can bind GL3 simultaneously, i.e., each of a pair of GL3 binding peptides binds a different site or epitope on GL3.

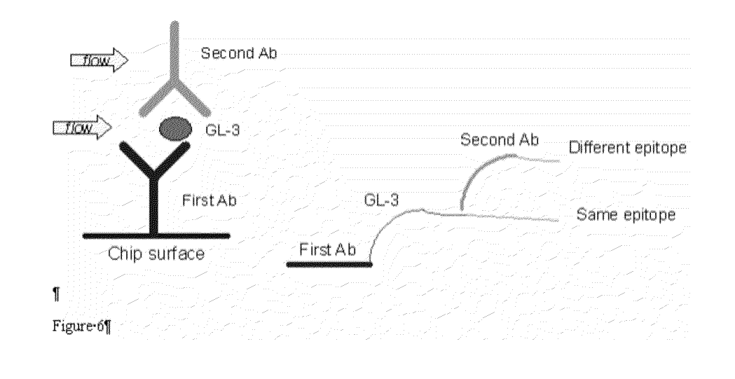

Surface plasmon resonance assays can be used as a quantitative method to measure binding between two molecules by the change in mass near an immobilized sensor caused by the binding of a first GL3 binding peptide from the aqueous phase to a GL3 molecule bound to a second GL3 binding peptide immobilized on a solid surface, e.g., a membrane on the sensor chip. This change in mass is measured as resonance units versus time after injection of a first GL3 binding peptide and is measured using a Biacore Biosensor (Biacore AB). The second GL3 binding peptide can be immobilized on the sensor chip (for example, research grade CM5 chip; Biacore AB) in a thin film lipid membrane according to methods described by Salamon et al. (Salamon et al., 1996, Biophys J. 71: 283-294; Salamon et al., 2001, Biophys. J. 80: 1557-1567; Salamon et al., 1999, Trends Biochem. Sci. 24: 213-219, each of which is incorporated herein by reference). Sarrio et al. demonstrated that SPR can be used to detect ligand binding to the GPCR A(1) adenosine receptor immobilized in a lipid layer on the chip (Sarrio et al., 2000, Mol. Cell. Biol. 20: 5164-5174, incorporated herein by reference). Conditions for assessing the binding of a GL3 binding peptide to GL3 bound to an immobilized second GL3 binding peptide, i.e., a sandwich, in an SPR assay can be fine-tuned by one of skill in the art using the conditions reported by Sarrio et al. and as described herein as a starting point. See FIG. 6. Similarly, pairs of GL3 binding peptides can be analyzed thus to assess their ability to simultaneously bind Lyso-GL3. Also, pairs of Lyso-GL3 binding peptides can be analyzed thus to assess their ability to simultaneously bind Lyso-GL3. Epitope mapping of GL3 binding peptides can be determined by alternative means well known to one of skill in the art.

### Sandwich Assays

As described above, in a typical sandwich method, after a fluid sample is reacted with an immobilized form of a GL3 binding peptide (primary reaction) and then reacted with a labeled form of a second GL3 binding peptide (secondary reaction), the activity of the labeling agent on the insoluble carrier is assayed from which the amount of GL3 in the fluid sample can be determined. The primary and secondary reactions may be carried out, simultaneously or sequentially with intervals. In the method of assaying the GL3 by the sandwich method, the GL3 binding peptide used for the primary reaction recognizes one site on GL3, while the GL3 binding peptide used for the secondary reaction preferably recognizes a different site on the GL3 molecule. In a preferred embodiment the primary and secondary GL3 binding peptides are BGR23 and GTC-1A, respectively. In a preferred embodiment the immobilized form of a GL3 binding peptide is attached to an ELISA plate, dipstick or an Immuno™ Stick (Nunc A/S), or a membrane for lateral flow assays or a biochip for surface plasmon resonance assays.

### Lateral Flow

As is known in the art, in a lateral flow device the sample, or an extract or dilution of said sample, which comprises the ligand(s) of interest, is permitted to flow laterally from the point of its application through one or more regions of one or more membrane surfaces to a detection zone. The presence of the ligand in the applied sample can be detected by a variety of protocols, including direct visualization of visible moieties associated with the captured ligand. A lateral flow device comprises a material capable of transporting a solution by capillary action, i.e., wicking Different areas or zones in the strip contain the reagents needed to produce a detectable signal as the ligand is transported to or through such zones.

When an applied aqueous sample comprising, or suspected to comprise, a ligand of interest contacts a first zone of the device, which contains a dry, reversibly immobilized, ligand specific reagent conjugate comprising a detectable label, the conjugate is reconstituted and mobilized, forming a first complex with ligand, if present. This first complex, together with mobilized, unbound labeled antibody, is capable of moving by capillary action to at least a second zone which is situated downstream of the first zone, where the first complex binds to ligand-specific reagent or antibody through the interaction of the ligand, resulting in the formation of a second, “sandwich” complex. Detection of this second complex can be detectable by any means suited to detection of the label, which is preferably a colored particle component, preferably by the naked eye, and indicates the presence of ligand in the sample. The detection of this complex can be measured, and used to quantitate the amount of ligand present in the sample.

In one embodiment the solid phase format of the lateral flow assay is cellulose acetate, cellulose, nitrocellulose or nylon. In a preferred embodiment, the solid phase format is nitrocellulose. In another embodiment, the solid phase format comprises a sample absorption pad, a strip of nitrocellulose and a bottom pad comprising a labeled anti-GL3 antibody.

As described herein, the methods of detecting a ligand in an aqueous solution, include the step of applying an aqueous sample solution to a device as described herein. In one exemplification of a sandwich lateral flow assay, sample is applied to a lateral flow device and results in the following series of events:

A) contacting a sample solution with a ligand-specific antibody conjugate containing a label, where the ligand-specific antibody conjugate is reversibly immobilized to a porous structure under conditions that allow mobilization of the ligand specific antibody conjugate upon contact with liquid, and the formation of a first complex in which the ligand is specifically bound to the ligand specific antibody conjugate;
B) as liquid sample carrying the first complex migrates down the structure from the point of application by capillary action, the sample subsequently contacts and binds to a second antigen specific antibody or protein which is irreversibly immobilized to the porous structure and located distal to the site where the ligand-specific antibody labeled conjugate reagent had been reversibly bound to the structure. The latter contacting occurs under conditions that permit the formation of a second complex in which the first complex is specifically bound to the second ligand specific antibody or protein, forming a sandwich where the ligand is bound by both the labeled antibody conjugate and the unlabeled, irreversibly bound antibody.
C) detecting the immobilized sandwich complex by detecting its colored particulate label component accumulated in the detection zone by a detection means appropriate to the nature of the particulate label, wherein detection of the third complex indicates the presence of the ligand in the aqueous solution.

The lateral flow device for use in an assay for detecting a ligand can be comprised of two or more test strips, each of which can comprise one or more porous components, membranes or filters which provides for capillary flow of a liquid sample. The device has a first pad, also called a conjugate pad, and a detection zone. This test strip is capable of wicking a fluid applied thereto by capillary action within the strip, from an upstream conjugate pad and into a downstream detection zone. The strip can have reagents deposited in zones along the longitudinal length of the membrane. Ligand in the sample contacts the reagents located within the test strip as the sample traverses the length of the strip. Test strip components, e.g. porous supports or membranes such as glass fiber filter and nitrocellulose are available from commercial suppliers or can be customized by laboratory personnel skilled in the art, or by a commercial immunodiagnostic supplier, to include immunoreagents specific for the ligand to be detected.

A variation of the test strip of a lateral flow assay uses what is commonly referred to as an immunostrip. An immunostrip is produced using membranes and filters through which a liquid sample is drawn by capillary action. The GL3 in the sample reacts with the antibodies contained in the immunostrip as it moves the length of the strip. To detect GL3 in a liquid sample, encompassing the GL3 sandwich, the liquid sample is added to the immunostrip. As the liquid sample migrates to the opposite end of the immunostrip, any GL3 in the sample reacts with labeled GL3 specific antibodies and is captured in a line containing an immobilized GL3 antibody. Detection of the signal on the test line indicates that GL3 is in the sample.

### Procedural Variations on the Assay

In one embodiment the reagents are combined in such a manner that the accumulation of detectable label at the immobilized GL3 binding peptide is inversely correlated to the concentration of GL3 in the sample applied to the assay. In one aspect, a sample fluid being analyzed with respect to its concentration of GL3 is incubated with an excess amount of a labeled GL3 binding peptide, and also incubated with a complex comprising exogenous GL3 (i.e., GL3 from a source other than the applied sample aliquot) bound to a GL3 binding peptide immobilized to a solid phase such as an immunostrip. Any labeled GL3 binding peptide which did not bind GL3 from the applied sample aliquot is available to bind the exogenous GL3 bound to the immobilized GL3 binding peptide. The quantity of the label bound to the immobilized GL3 is measured to determine the amount of the antigen in the sample fluid, which bears an inverse correlation to the amount of label detected as being bound to the immobilized GL3. That is, the more GL3 present in the sample, the less labeled GL3 binding peptide will be detected bound to the immobilized GL3.

This assay format can be applied to a lateral flow assay where sample is applied to a membrane on to which a labeled GL3 binding peptide is reversibly attached, up stream from the placement of an irreversibly attached GL3-binding peptide which is complexed with GL3 from an exogenous source. As the sample flows by diffusion down the membrane, any GL3 in the sample will bind the reversibly attached labeled GL3 binding peptide. The migration of the liquid sample wicking down the membrane releases the reversibly bound, labeled GL3 binding peptide, regardless if it bound GL3 in the sample. The sample now containing the previously attached labeled GL3 binding peptides, continues to migrate down the membrane, contacting the irreversibly immobilized GL3 binding peptide bound to the exogenous GL3. Any of the previously attached labeled GL3 binding peptides which did not bind GL3 from the sample is available to bind the irreversibly immobilized GL3 binding peptide bound to the exogenous GL3, forming a sandwich. The formation of a sandwich is detected and optionally measured through the labeled GL3 binding peptide component of the sandwich. This measurement is inversely correlated with the amount of GL3 in the applied sample aliquot, and can be used to calculate the concentration of GL3 in the sample. Additionally, a GL3 binding peptide which is unoccupied by GL3 can be irreversibly attached at a site further downstream from the first irreversibly immobilized complex. As the sample continues its migration and encounters this latter irreversibly attached GL3 binding peptide which is unoccupied by GL3, any complexes comprising GL3 from the sample and the labeled GL3 binding peptide formed near the beginning of the sample migration can bind the irreversibly attached GL3 binding peptide, forming a detectable GL3 sandwich which can be measured. This measurement directly correlates with the amount of GL3 present in the sample.

### Non Sandwich Assays

In one embodiment, an assay is provided where the GL3 in a sample fluid is reacted in a primary reaction with an excess amount of a labeled form of a GL3 binding peptide, followed by incubation in a secondary reaction with GL3 immobilized to a solid phase. In the secondary reaction, any labeled GL3 binding peptide which has not bound to GL3 in the sample in the primary reaction is available to bind the immobilized GL3. The quantity of the label bound to the immobilized GL3 is measured to determine the amount of the antigen in the sample fluid, which bears an inverse correlation to the amount of label detected as being bound to the immobilized GL3. That is, the more GL3 present in the sample, the less labeled GL3 binding peptide will be detected bound to the immobilized GL3. This assay can be adapted to various surfaces as described above.

### Immunoassay Kit

The materials for use in the assay of the invention are ideally suited for the preparation of a kit. An immunoassay kit for the detection of GL3 in a sample contains one or more of the GL3 binding peptides as described above. In one embodiment of a kit, one or more of the GL3 binding peptides is immobilized onto a solid surface. In one embodiment of the kit, the GL3 binding peptide is bound to GL3. In one embodiment of the kit GL3 itself is bound to a surface. Any of the reagents may be bound to the surface directly or indirectly, and reversibly or irreversibly. The solid surface can be any suitable surface for carrying out the assays described within, including, but not limited to a membrane, a dipstick, an Immuno™ Stick (Nunc A/S), a chip, and a suitable surface molded into a desirable shape such as a well or a paddle. The kit may additionally contain equipment for obtaining the sample, a vessel for containing the reagents, a timing means, a buffer for diluting the sample, and a calorimeter, reflectometer, or standard against which a color change may be measured. The kit may include the reagents in the form of an immunostrip as described above.

In a preferred embodiment, the reagents, including the GL3 binding peptides are dry. Addition of liquid sample to the vial or strip or an Immuno™ Stick (Nunc A/S) results in solubilization of the dry reagent, rendering it functional.

Such a kit may comprise a carrier means being compartmentalized to receive in close confinement one or more container means such as vials, tubes, and the like, each of the container means comprising one of the separate elements to be used in the method. For example, one of the container means may comprise one or more GL3 binding peptides, including for example, a pair of GL3 binding peptides which bind GL3 simultaneously, e.g., GTC-1A and BGR23. One or more of the GL3 binding peptides is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label.

The antibodies are collectively assembled in a kit with conventional immunoassay reagents for detection of the GL3 using the immunoassay described herein. The kit may optionally contain both monoclonal and polyclonal antibodies and a standard for determining the presence of the GL3 in a sample. The kit containing these reagents provides for simple, rapid, on site detection of the protein.

The invention also provides a kit for the detection and quantification by the immunoassay method comprising: a) a means of extracting the GL3 from a sample; b) a solid support comprising a primary anti-GL3 antibody bound to the solid support; c) a secondary anti-GL3 antibody; and d) a detection antibody capable of immunologically binding to the secondary antibody and wherein the detection antibody is labeled with a means of detection.

The rapid, sensitive, and specific assays for analyzing GL3 and LysoGL3 described herein should prove useful in the development and evaluation of various therapeutic strategies such as enzyme and gene replacement for the treatment of Fabry disease. It also may be useful in examining the role of GL3 and LysoGL3 in various biological processes and disorders in addition to Fabry's disease, such as cell growth, B cell differentiation and apoptosis, cancer, α-interferon signaling, interleukin-1β, tumor necrosis factor-b, hemolytic uremic syndrome, and familial dysautonomia, the involvement of GL3 and/or LysoGL3 being reported by Zeidner et al. Analytical Biochemistry (1999) 267, 104-113.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

The reagents, immunoassay methods, and kits described above will be further understood with reference to the following non-limiting examples. The working examples below show typical experimental protocols and reagents that can be used in the detection of GL3 in samples such as urine. Such examples are provided by way of illustration and not by way of limitation. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### WORKING EXAMPLES

### Example 1

### Traditional ELISA

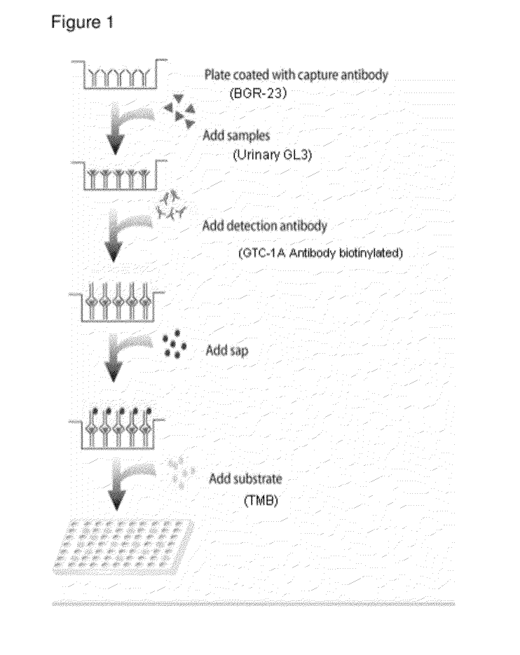

The GL3 traditional sandwich ELISA is based upon an IgG (monoclonal) coating antibody and an IgM (monoclonal) detector Ab. The pairing with BGR-23 (coated) and biotinylated GTC-1A (detector) demonstrated that the system could measure GL3 in urine. Steps of addition: 1. The ELISA plate is coated with BGR-23 IgG antibody. 2. Urine samples are added. 3. Biotin labeled GTC-1A IgM detector antibody is added. 4. Streptavidin peroxidase is added. 5. TMB develops the assay. FIG. 1 illustrates an outline of the assay.

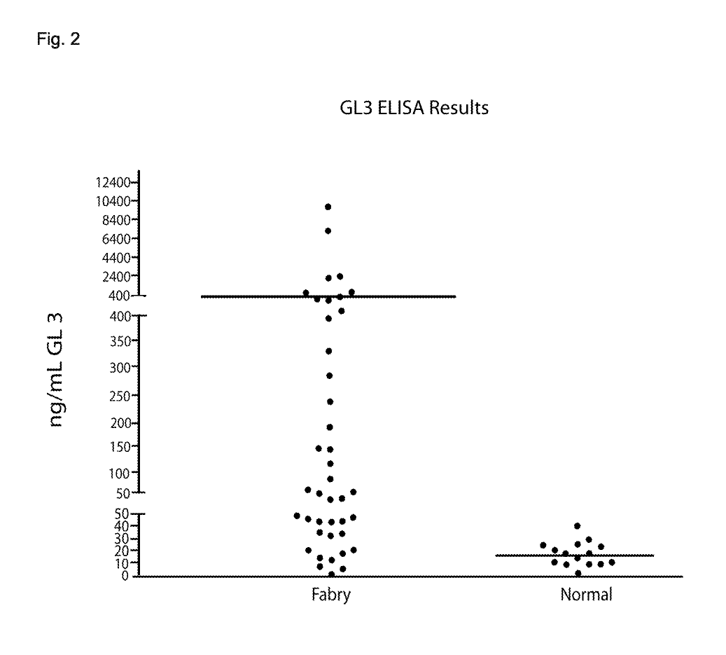

Fabry sample data (40 samples, it should be noted that many Fabry positive patients were on treatment) was generated using Matreya reconstituted GL3 (standard). A set of normal samples (15 in house) were used for comparison. The range of concentrations measured in the Fabry positive samples was ng/mL-10734 ng/mL. The 95% confidence interval range for Fabry positive samples was 188-1483 ng/mL. The range of concentrations measured in the Normal Samples was 0 ng/mL-40 ng/mL. The 95% confidence interval range for Normal Samples was 11-25 ng/mL. The data is presented in FIG. 2.

Data generated from the GL3 ELISA indicated that a population of Fabry samples can be distinguished from normal samples.

### Example 2

### Rapid ELISA

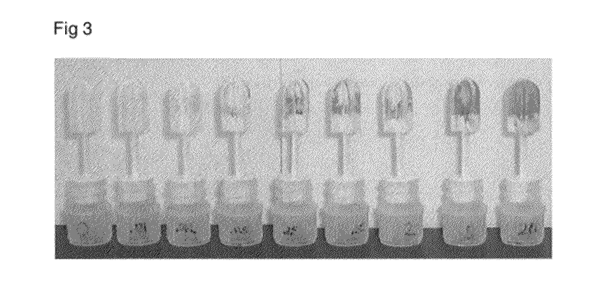

Using the GL3 binding proteins described in Example 1, a rapid, semi-quantitative assay that measures globotriaosylceramide in whole urine samples (rapid ELISA) was developed. The test system consists of a tube and an Immuno™ Stick (Nunc A/S) (stick with paddle)(ThermoScientific). The polypropylene tubes provide a low protein binding material necessary for minimizing any cross-reactivity resulting from the various reactions of the assay; while the polystyrene paddles provide an ideal coating material for protein attachment (similar to a microtiter well). The paddles were uniformly coated with a commercially available IgG2b antibody (BGR23) specific for globotriaosylceramide. A urine sample containing concentrations of GL-3 of 0, 156, 312, 625, 1250, 2500, 5000, 10000, or 20000 ng/mL was then added to the tube and incubated with the paddle. Biotin labeled GTC-1A antibody specific for globotriasylceramide was then added to the tube and incubated with the paddle, followed by the addition of a dilution of peroxidase labeled Strepavidin used to detect the biotin labeled antibody. Finally, 3,3′,5,5′ tetramethylbenzidine (TMB) substrate was added to the tube and incubated with the paddle to generate a negative (no color) result or positive (blue colored paddle) result. The data in FIG. 3 show an increase in color of the paddles with increasing amounts of GL3 in the samples.

### Example 3

### Inhibition Assay

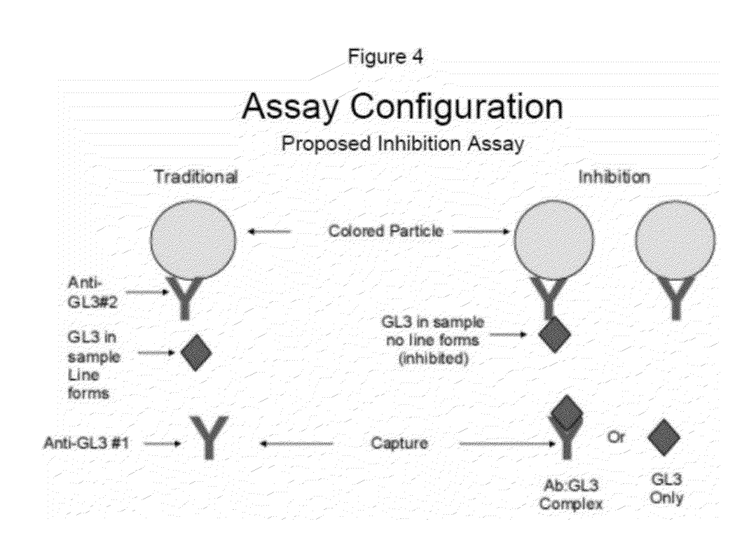

The sandwich assay which formed the basis of the assays described in Examples 1 and 2 was developed into an inhibition assay. In contrast to the traditional assays described in Examples 1 and 2, where an increase in the detected signal correlated with an increase in GL3 in the sample, the following inhibition assays display an inverse correlation between the concentration of GL3 in the sample and the signal detected. The traditional assay and the inhibitor assay are compared in the illustration presented in FIG. 4. In each assay, the above sandwich assay is applied to a lateral flow assay. The GL-3 concentration of the tested samples is given in the text.

In the traditional lateral flow assay, diagrammed on the left side of FIG. 4, the capture GL3 binding polypeptide is not occupied with GL3. When the sample applied to the lateral flow assay is added, it first encounters a labeled GL3 binding peptide and any GL3 present in the sample forms a complex with the labeled GL3 binding peptide. This complex then migrates to the location of the fixed capture GL3 binding polypeptide, forming a second complex. This second complex comprising GL3 bound to each of the GL3 binding peptides, i.e., the labeled GL3 binding peptide and the fixed, capture GL3 peptide. Each of the two GL3 binding peptides is distinct and binds to a distinctly different portion of the GL3 molecule. The amount of labeled antibody detected at the location of the capture antibody directly correlates with the amount of GL3 in the applied sample.

In the inhibition lateral flow assay, diagrammed on the right side of FIG. 4, before application of the sample, the capture GL3 binding polypeptide is occupied with GL3. When the sample applied to the lateral flow assay is added, it first encounters a labeled GL3 binding peptide and any GL3 present in the sample forms a complex with the labeled GL3 binding peptide. This complex then migrates to the location of the fixed capture GL3 binding polypeptide where it is unable to form a second complex with the capture GL3 binding peptide which was previously loaded with GL3. Thus, in samples containing GL3, the GL3 detector antibody complex is not captured by the loaded capture antibody and no label accumulates at the site of the capture antibody. Accordingly, the amount of labeled antibody detected at the location of the capture antibody display an inverse correlation with the amount of GL3 in the applied sample.

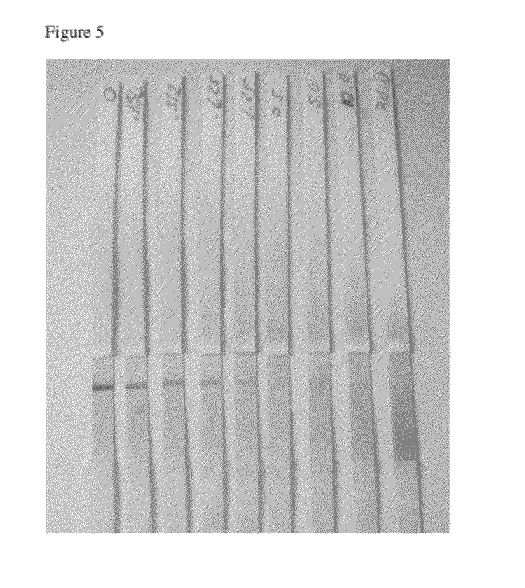

FIG. 5 displays the results of a GL3 inhibition Lateral Flow assay designed to test the limits of this assay in detecting GL3 detection in a buffer matrix. A series of dilutions of GL3 in concentrations ranging from 0 to 20 ug/ml (0.156, 0.312, 0.625, 1.25, 2.5, 5.0, 10, and 20 ug/ml, respectively) were run on a lateral flow assay described above. In this lateral flow assay, the capture antibody complex comprised GL3 and the IgG2 GL3 specific antibody BGR23. The detector antibody was the IgM GL3 specific antibody GTC-1A conjugated to Carbon-sol particles. The assay time was 10-20 minutes, (two step liquid conjugate). This sensitive assay was able to detect a difference between a negative control and a sample containing 0.156 ug/ml.

### Example 4

### Rapid Epitope Mapping of GL3 Antibodies Using Biacore

One requisite of the sandwich assays described herein is that the site to which each of the pair of GL3 binding peptides bind to GL3 be different from each other. Thus potential pairs of GL3 specific binding peptides can be assessed with respect to the binding sites on GL3 as well as to their relative specificity and affinity. The Biacore assay not only provides for a rapid comparison of epitope sites bound by various GL3 binding peptides, including GL3 antibodies, but also provides a means to screen for potential pairs of GL3 binding peptides to use in the sandwich assays described herein.

The Biacore was used to set up an Ab-Ag-Ab sandwich experiment by immobilizing each antibody to a biosensor surface, injecting GL3 over all 4 flow cells, then flowing antibody over the captured lipid. FIG. 6 shows a schematic of this experiment, with a comparison of the divergent results produced when the two antibodies bind the same sites and/or impede the binding of the second GL3 antibody vs. when the two GL3 antibodies bind different sites and do not significantly impede each other's binding. A binding response would be observed if the epitope is different from the antibody immobilized on the surface. No response would be detected if the site on GL3 is already being occupied (for example, in FIG. 6, Ab1 should show no response to GL3 binding to immobilized Ab1).

In a first step a liquid sample containing GL3 is added to an immobilized first GL3 binding peptide (antibody), and in a second step a liquid solution containing a second GL3 binding peptide (antibody) is added. If the second GL3 binding peptide binds an accessible site on GL3 bound to the immobilized first GL3 binding peptide, then a sandwich assay is formed. If the second GL3 binding peptide does not bind an accessible site on GL3 bound to the immobilized first GL3 binding peptide, (as in the case where the first and second GL3 binding peptides bind the same epitope), then no sandwich assay is formed. So an increase in resonance units is generated with the binding of GL3 to the immobilized first GL3 binding peptide, followed by a further increase if a second GL3 binding peptide binds the GL3 bound to the immobilized first GL3 binding peptide, forming a GL3 sandwich. If no GL3 sandwich is formed, then no further increase in resonance units is generated.

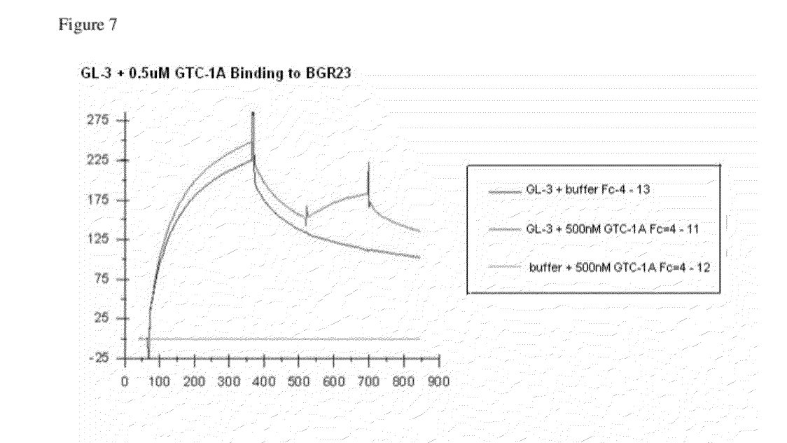

BGR23 antibody to GL3 was coupled to a CM5 chip using amine chemistry, (14104RU BGR23 diluted to 50 ug/ml in acetate pH 4.5. GL3 was diluted to 20 uM in HBSEP buffer (10 mM HEPES, pH 7.4, 150 mM NaCl, 3 mM EDTA, 0.005% P20) and injected into the flow cell for 5 minutes, followed by injection of 500 nM GTC-1A for three minutes to the bound GL3. After regeneration, antibody alone was injected for a reference subtraction in addition to buffer controls. The results show clear binding of 500 nM GTC-1A (top curve shown in FIG. 7) to GL3 bound to immobilized BGR23. These results suggest that the antibodies GTC-1A and BGR23 bind to different epitopes on GL3, and thus are useful in designing assays for GL3.

### Example 5

### Weak Binding of Lyso-GL3 to BGR23 Mouse IgG2b

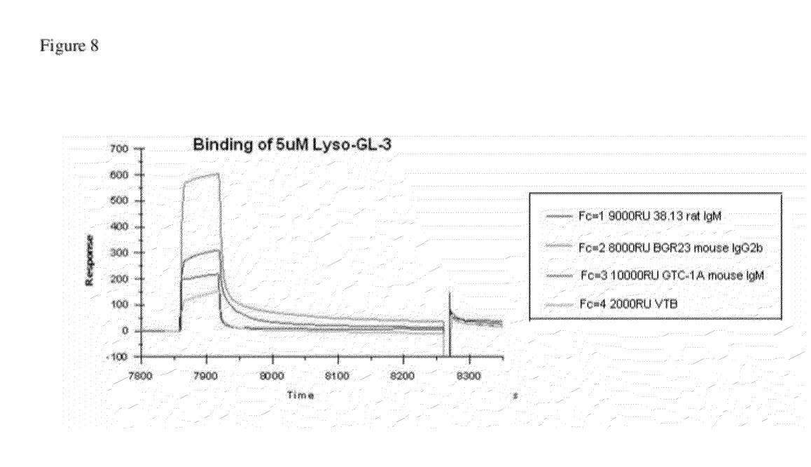

In addition to GL3, the above Biacore assay can be used to screen for binding peptides capable of detecting Lyso GL3. As described herein, Lyso GL3 is a by product of GL3, and also is found in urine, blood, blood plasma, and other tissues at a higher concentration in people with Fabry's disease or having a defective alpha-galactosidase A, relative to normal individuals. Approximately 9000RU Ab1 38.13 rat IgM, ˜8000RU Ab2 BGR23, and 10,000RU Ab3 GTC-1A was immobilized using amine chemistry to flow cells 1, 2, and 3, respectively. Lyso-GL3 was diluted to 504 and injected over each flow cell. Lyso GL3 showed low affinity to immobilized BGR23. Lyso-GL3 displayed very weak binding to the remaining immobilized antibodies (GTC-1A and 38-13) and to immobilized beta subunit of Verotoxin. See FIG. 8.

### Example 6

### Verotoxin Beta Subunit (VTB) Displayed Clear Binding to 504 GL3 Captured to Immobilized BGR23 in a Biacore Assay

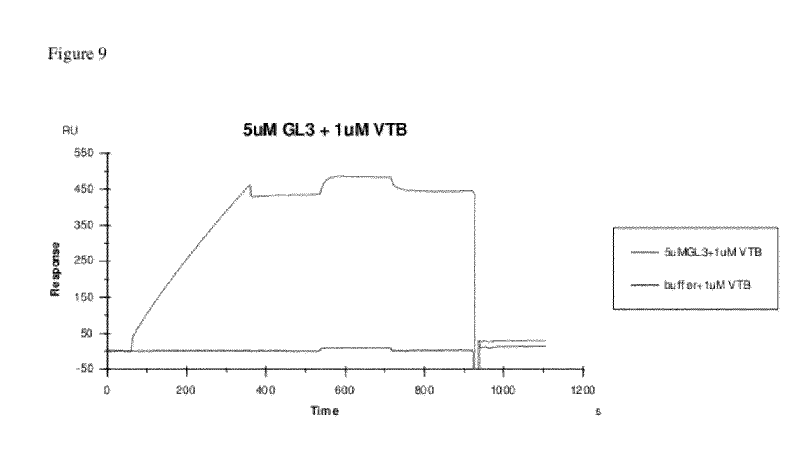

GL3 binding peptides other than antibodies can also be used in the GL3 antibody sandwich described herein. In a Biacore assay, 10,000RU Ab GTC-1A was immobilized using amine chemistry to a flow cell, and 504 GL3 injected over the flow cell with the immobilized GTC-1A antibody. VTB at a concentration of 104 was then injected onto the flow cell. Clear binding of VTB to GL3 captured by the immobilized GTC-1A antibody was observed, see FIG. 9.

The results indicate that Verotoxin beta subunit (VTB) displayed binding to 5 μM GL3 captured by immobilized BGR23 in a Biacore Assay. The formation of a GL3 sandwich suggests that VTB binds to a different binding epitope on GL3 from BGR23, and thus are useful in designing assays for GL3. See FIG. 9.

### Example 7

### Verotoxin Beta Subunit (VTB) Displayed Binding to 5 μM Lyso-GL3 Captured to Immobilized BGR23 in a Biacore Assay

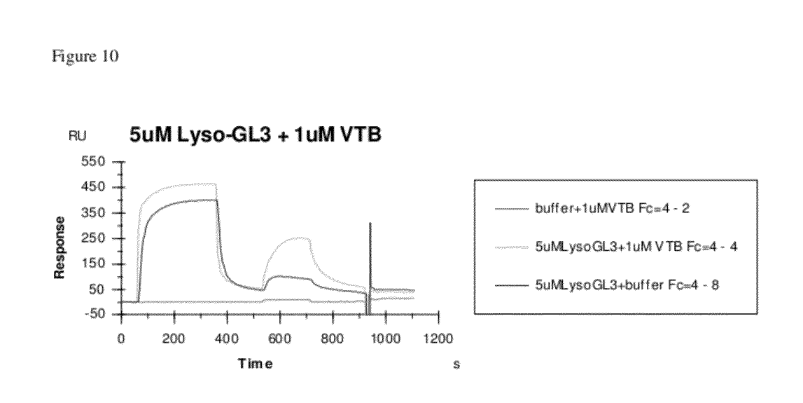

A Biacore assay was used to screen for peptides other than antibody peptides, capable of binding to Lyso-GL3 bound to an immobilized first Lyso-GL3 binding peptide. VTB appears to bind the captured Lyso-GL3, even though the binding affinity is weak. In a Biacore assay, 10,000RU Ab3 GTC-1A was immobilized using amine chemistry to a flow cell, and 5 μM Lyso-GL3 injected over the flow cell with the immobilized GTC-1A antibody. VTB at a concentration of 1 μM was then injected onto the flow cell. Binding of VTB to Lyso-GL3 captured by the immobilized GTC-1A antibody was observed, see FIG. 10.

The results indicate that Verotoxin beta subunit (VTB) displayed binding to 5 μM Lyso-GL3 captured by immobilized BGR23 in a Biacore Assay. The formation of a Lyso-GL3 sandwich suggests that VTB binds to a different binding epitope on Lyso-GL3 from BGR23, and thus are useful in designing assays for Lyso-GL3. See FIG. 10.

1. A method for detecting GL3 in a sample, wherein said GL3 comprises a first and a second binding site, said method comprising;
(A) contacting GL3 in said sample with a first GL3 binding peptide that specifically binds said first binding site, under conditions which provide for formation of a first complex comprising said first GL3 binding peptide and said GL3,
(B) contacting said first complex with a second GL3 binding peptide that specifically binds said second binding site, under conditions which provide for formation of a second complex comprising said first complex and said second GL3 binding peptide, and
(C) detecting said second complex,
wherein detection of said second complex is indicative of GL3's presence in said sample.

2. The method of claim 1, wherein said second GL3 binding peptide is immobilized to a surface.

3. The method of claim 2, wherein said first GL3 binding peptide comprises a label.

4. The method of claim 2, further comprising quantitating the level of GL3 in said sample.

5. The method of claim 2, wherein said first GL3 binding peptide is an antibody, or a fragment thereof.

6. The method of claim 5, wherein said antibody is a monoclonal antibody

7. The method of claim 5, wherein said antibody is GTC-1A.

8. The method of claim 2, wherein said second GL3 binding peptide is an antibody, or a fragment thereof.

9. The method of claim 8, wherein said antibody is a monoclonal antibody

10. The method of claim 9, wherein said monoclonal antibody is BGR23.

11. The method of claim 2, wherein said surface is a solid and said method of detection is ELISA.

12. The method of claim 2, wherein said surface is a membrane and said method comprises a lateral flow device.

13. The method of claim 3, wherein said label is selected from radioactivity, a chemiluminescent label, a fluorescent label, a colored particle, sol, gold, and carbon beads.

14. The method of claim 2, wherein said sample is urine or plasma.

15. The method of claim 14, wherein said sample is a urine sample of a patient suspected of having Fabry's disease and wherein a level of GL3 detected in said sample which is at least 2 fold higher than that in a healthy control is indicative of Fabry's disease.

16. A method of detecting Fabry's disease in a patient comprising assaying the level of GL3 in a urine or plasma sample of said patient, wherein said GL3 comprises a first and a second binding site, said method comprising;
(A) incubating said sample with a first GL3 binding peptide that specifically binds said first binding site, under conditions which provide for formation of a first complex comprising said first GL3 binding peptide and said GL3,
(B) contacting said first complex with a second GL3 binding peptide that specifically binds said second binding site, under conditions which provide for formation of a second complex comprising said first complex and said second GL3 binding peptide, and
(C) detecting and quantitating said second complex,
wherein the amount of second complex detected in step (C) reflects the level of GL3 in said sample, and wherein a level of GL3 detected in said sample which is at least 2 fold higher than that of a healthy control is indicative of Fabry's disease.

17. A method of monitoring the efficacy of therapeutic treatment of Fabry's disease in a patient, comprising assaying the level of GL3 in a urine or plasma sample of said patient, wherein said GL3 comprises a first and a second binding site, comprising;
(A) incubating said sample with a first GL3 binding peptide that specifically binds said first binding site, under conditions which provide for formation of a first complex comprising said first GL3 binding peptide and said GL3,
(B) contacting said first complex with a second GL3 binding peptide that specifically binds said second binding site, under conditions which provide for formation of a second complex comprising said first complex and said second GL3 binding peptide, and
(C) detecting and quantitating said second complex,
wherein the amount of second complex detected in step (C) reflects the level of GL3 in said sample, and wherein a decrease in concentration GL3 in said sample relative to that in a previous sample of said patient indicates said treatment of Fabry's disease in said patient is efficacious.

18. A method for detecting GL3 in a sample wherein said GL3 comprises a first and a second binding site, said method comprising:
(A) providing a first complex comprising GL3 bound to a first GL3 binding peptide immobilized on a surface, wherein said first GL3 binding peptide specifically binds said first binding site, comprising the steps of:
(i) immobilizing said first GL3 binding peptide to said surface,
(ii) incubating said first GL3 binding peptide with GL3 under conditions which provide for formation of a first complex comprising said first GL3 binding peptide bound to GL3 at said first site;
(B) incubating said sample with a second GL3 binding peptide, wherein said second GL3 binding peptide specifically binds said second binding site, under conditions which provide for formation of a second complex comprising said second binding peptide and GL3 from said sample and;
(C) incubating the components of step (B) with said first complex, under conditions which provide for the formation of a third complex comprising said first complex and said second GL3 binding peptide; and
(D) detecting said third complex, if present,
wherein a lack of detection of said third complex in step (D) indicates the presence of GL3 in said sample.

19. The method of claim 18, wherein said first GL3 binding peptide is irreversibly immobilized on a surface.

20. The method of claim 18, wherein said second GL3 binding peptide comprises a label.

21. The method of claim 18 further comprising quantitating the level of GL3 in said sample using control samples containing known amounts of GL3, wherein the level of GL3 in said sample is inversely correlated to the level of said third complex detected in step (D).

22. The method of claim 18, wherein said first GL3 binding peptide is an antibody, or a fragment thereof.

23. The method of claim 22, wherein said antibody is a monoclonal antibody

24. The method of claim 23, wherein said monoclonal antibody is BGR23.

25. The method of claim 18, wherein said second GL3 binding peptide is an antibody, or a fragment thereof.

26. The method of claim 25, wherein said antibody is a monoclonal antibody

27. The method of claim 25, wherein said antibody is GTC-1A.

28. The method of claim 18, wherein said surface is a solid and said method of detection is ELISA.

29. The method of claim 18, wherein said surface is a membrane and said method comprises lateral flow.

30. The method of claim 20, wherein said label is selected from radioactivity, a chemiluminescent label, a fluorescent label, a colored particle, sol, gold, and carbon beads.

31. The method of claim 18, wherein said sample is urine or plasma.

32. The method of claim 18, wherein said sample is urine.

33. The method of claim 21, wherein said sample is a urine sample of a patient suspected of having Fabry's disease, and wherein a concentration of GL3 detected in said sample which is at least 2 fold higher than that of a healthy control is indicative of Fabry's disease in said patient.

34. A method of detecting Fabry's disease in a patient comprising assaying the level of GL3 in a urine or plasma sample of said patient, wherein said GL3 comprises a first and a second binding site, comprising;
(A) providing a first complex comprising GL3 bound to a first GL3 binding peptide immobilized on a surface, wherein said first GL3 binding peptide specifically binds said first binding site, comprising the steps of:
(i) immobilizing said first GL3 binding peptide to said surface,
(ii) incubating said first GL3 binding peptide with GL3 under conditions which provide for formation of a first complex comprising said first GL3 binding peptide bound to GL3 at said first site;
(B) incubating said sample with a second GL3 binding peptide, wherein said second GL3 binding peptide specifically binds to said second binding site, under conditions which provide for formation of a second complex comprising said second binding peptide and GL3 from said sample and;
(C) incubating the components of step (B) with said first complex, under conditions which provide for the formation of a third complex comprising said first complex and said second GL3 binding peptide; and
(D) detecting and quantitating said third complex, if present,
wherein a lack of detection of said third complex in step (D) indicates the presence of GL3 in said sample, wherein the concentration of GL3 in said sample is inversely correlated to the amount of said third complex detected, and wherein a level of GL3 detected in said sample which is at least 2 fold higher than that of a healthy control is indicative of Fabry's disease in said patient.

35. A method of monitoring the efficacy of therapeutic treatment of Fabry's disease in a patient, comprising assaying the level of GL3 in a urine or plasma sample of said patient, wherein said GL3 comprises a first and a second binding site, comprising;
(A) providing a first complex comprising GL3 bound to a first GL3 binding peptide immobilized on a surface, wherein said first GL3 binding peptide specifically binds said first binding site, comprising the steps of:
(i) immobilizing said first GL3 binding peptide to said surface,
(ii) incubating said first GL3 binding peptide with GL3 under conditions which provide for formation of a first complex comprising said first GL3 binding peptide bound to GL3 at said first site;
(B) incubating said sample with a second GL3 binding peptide wherein said second GL3 binding peptide specifically binds to said second binding site, under conditions which provide for formation of a second complex comprising said second binding peptide and GL3 from said sample and;
(C) incubating the components of step (B) with said first complex, under conditions which provide for the formation of a third complex comprising said first complex and said second GL3 binding peptide; and
(D) detecting and quantitating said third complex, if present,
wherein a lack of detection of said third complex in step (D) indicates the presence of GL3 in said sample, wherein the level of GL3 in said sample is inversely correlated to the amount of said third complex detected, and wherein a level of GL3 detected in said sample which is less than that of a previous sample of said patient indicates said treatment of Fabry's disease in said patient is efficacious.

36. A method for detecting GL3 in a sample comprising;
(A) immobilizing GL3 to a surface,
(B) incubating said sample with a GL3 binding peptide under conditions which provide for formation of a complex comprising GL3 from said sample and said GL3 binding peptide,
(C) incubating the components of step (B) with the immobilized GL3 of step (A), under conditions which provide for the formation of a second complex comprising said immobilized GL3 of step (A) and said GL3 binding peptide, and
(D) detecting said second complex, if present
wherein no detectable said second complex in step (D) indicates the presence of GL3 in said sample.

37. The method of claim 36, wherein said GL3 binding peptide is labeled.

38. The method of claim 36 further comprising quantitating the level of GL3 in said sample using control samples containing known amounts of GL3, and wherein the level of GL3 in said sample is inversely correlated to the amount of said second complex detected in step (D).

39. The method of claim 36, wherein said GL3 binding peptide is an antibody, or a fragment thereof.

40. The method of claim 39, wherein said antibody is a monoclonal antibody.

41. The method of claim 40, wherein said monoclonal antibody is BGR23.

42. The method of claim 36, wherein said surface is a solid and said method of detection is ELISA.

43. The method of claim 36, wherein said surface is a membrane and said method comprises lateral flow.

44. The method of claim 37, wherein said label is selected from radioactivity, a chemiluminescent label, a fluorescent label, a colored particle, sol, gold, and carbon beads.

45. The method of claim 36, wherein said sample is urine or plasma.

46. The method of claim 36, wherein said sample is of a patient suspected of having Fabry's disease and wherein a 200 percent increase of GL3 in said sample relative to that in a healthy control is indicative of Fabry's disease.

47. A method of detecting Fabry's disease in a patient comprising assaying the level of GL3 in a urine or plasma sample of said patient comprising;
(A) immobilizing GL3 to a surface,
(B) incubating said sample with a GL3 binding peptide under conditions which provide for formation of a complex comprising GL3 from said sample and said GL3 binding peptide,
(C) incubating the components of step (B) with the immobilized GL3 of step (A), under conditions which provide for the formation of a second complex comprising said immobilized GL3 of step (A) and said GL3 binding peptide, and
(D) detecting said second complex, if present
wherein no detectable said second complex in step (D) indicates the presence of GL3 in said sample, wherein the level of GL3 in said sample is inversely related to the amount of said second compound detected in step (D), and wherein a 200 percent increase of GL3 in said sample relative to that in a healthy control is indicative of Fabry's disease.

48. A method of monitoring the efficacy of therapeutic treatment of Fabry's disease in a patient, comprising assaying the level of GL3 in a urine or plasma sample of said patient, comprising;
(A) immobilizing GL3 to a surface,
(B) incubating said sample with a GL3 binding peptide under conditions which provide for formation of a complex comprising GL3 from said sample and said GL3 binding peptide,
(C) incubating the components of step (B) with the immobilized GL3 of step (A), under conditions which provide for the formation of a second complex comprising said immobilized GL3 of step (A) and said GL3 binding peptide, and
(D) detecting said second complex, if present
wherein no detectable said second complex in step (D) indicates the presence of GL3 in said sample, wherein the level of GL3 in said sample is inversely related to the amount of said second compound detected in step (D), and wherein a level of GL3 detected in said sample which represents a decrease in concentration GL3 in said sample relative to that in a previous sample of said patient indicates said treatment of Fabry's disease in said patient is efficacious.

49. A method of screening for a pair of GL3 binding peptides that bind GL3 simultaneously in a sandwich format comprising:
(i) contacting a liquid sample containing GL3 to an immobilized first GL3 binding peptide (antibody), and
(ii) contacting a liquid solution containing a second GL3 binding peptide to the components of step (i) (antibody),
(iii) detecting the presence of a GL3 sandwich comprising said second GL3 binding peptide bound to said GL3 captured by said immobilized first GL3 binding peptide,
wherein detection of a GL3 sandwich in step (iii) indicates that said first GL3 binding peptide and said second GL3 binding peptide represent a pair of GL3 binding peptides that bind GL3 simultaneously in a sandwich format.

50. The method of claim 50, wherein said first GL3 binding peptide and/or said second GL3 binding peptide is a monoclonal antibody.

51. A kit comprising reagents designed to detect the presence of GL3 in a sample, wherein said kit comprises a pair of GL3 binding peptides that bind GL3 simultaneously in a sandwich format.
